(19) United States
(12) Patent Application Publication
(10) Pub. No.: US 2022/0175672 A1
(43) Pub. Date: Jun. 9, 2022

(54) COMPOSITIONS AND METHODS FOR SUSTAINED DRUG RELEASE FROM AN INJECTABLE HYDROGEL

(71) Applicant: Contraline, Inc., Charlottesville, VA (US)

(72) Inventors: Gregory Grover, Houston, TX (US); Kevin Eisenfrats, Charlottesville, VA (US)

(21) Appl. No.: 17/677,295

(22) Filed: Feb. 22, 2022

Related U.S. Application Data
(63) Continuation of application No. PCT/US20/47642, filed on Aug. 24, 2020.
Application No. PCT/US20/47642 is a continuation of application No. PCT/US21/32235, filed on May 13, 2021.
(60) Provisional application No. 63/056,124, filed on Jul. 24, 2020.
(60) Provisional application No. 62/890,409, filed on Aug. 22, 2019.
(60) Provisional application No. 63/056,124, filed on Jul. 24, 2020.
(60) Provisional application No. 63/024,628, filed on May 14, 2020.

(51) Int. Cl. A61K 9/06; A61K 47/60; A61K 9/00; A61K 31/568; A61K 31/506; A61K 31/675; A61K 31/65; A61P 31/04; A61P 31/12; A61P 15/16; A61P 15/18

(52) U.S. Cl. CPC A61K 9/06; A61K 47/60; A61K 9/0024; A61K 31/568; A61K 31/506; A61P 15/18; A61K 31/65; A61P 31/04; A61P 31/12; A61P 15/16; A61K 31/675

(57) ABSTRACT

Compositions, devices and methods of using one or more hydrogel for contraception as well as localized, sustained delivery of drugs are disclosed. Device (e.g., hydrogel) embodiments are easily injectable, have a quick gelation rate, are highly durable, and are capable of lasting greater than 3 months in vivo. The devices/hydrogel may be used for occlusion of a bodily duct, such as the vas deferens and/or fallopian tubes, for male and female contraception, respectively. Once implanted, the device/hydrogel is able to release one or more drugs, such as small molecules or biologics, indirectly, systemically, and/or directly to the site of interest over a sustained period of time, such as for prevention and/or treatment of STIs.

### CROSS-REFERENCE TO RELATED APPLICATIONS

The present application is a Continuation application of International Application No. PCT/US20/47642, filed Aug. 24, 2020, which application relies on the disclosure of and claims priority to and the benefit of the filing dates of U.S. Provisional Patent Application Nos. 63/056,124, filed Jul. 24, 2020 and 62/890,409, filed Aug. 22, 2019. The present application is a Continuation application of International Application No. PCT/US21/32235, filed May 13, 2021, which application relies on the disclosure of and claims priority to and the benefit of the filing date of U.S. Provisional Patent Application Nos. 63/024,628, filed May 14, 2020 and 63/056,124, filed Jul. 24, 2020. Each application is hereby incorporated by reference herein in its entirety.

### BACKGROUND OF THE INVENTION

### Field of the Invention

The present invention relates to the fields of contraception and prevention of sexually transmitted infections or diseases. Provided are compositions, devices and methods of using one or more hydrogel for contraception as well as delivery of drugs.

Contraceptive usage has increased globally, yet 40-45% of pregnancies are unintended. In the United States alone, mistimed, unplanned or unwanted pregnancies result in a total public expenditure of ˜$21 billion per year. These pregnancies are considered high-risk with evidence that both the women and the children experience worse health outcomes than intended pregnancies. Popular contraceptive methods include male/female condoms, female hormone treatments (pills, patches, implants, etc.), and intrauterine devices. Over the past three decades, even though many effective female contraceptive options have been introduced to market, unintended pregnancy rates have remained relatively constant.

A promising strategy to reduce the number of unintended pregnancies and the subsequent societal and family costs is to increase contraceptive options, specifically options available to men. While men make up approximately 50% of the population, they currently only have three contraceptive options available to them: condoms, withdrawal, and vasectomy. When used correctly, condoms can be an effective contraceptive and prophylactic. However, condoms are frequently used incorrectly and are only approximately 85% effective overall, and have a low satisfaction rate. Vasectomy is considered permanent and very effective; however, it is difficult to reverse. The withdrawal method is the least effective form of birth control, but from 2002-2015 withdrawal use has doubled while vasectomy (permanent) and condoms (single use at time of need) usage did not significantly increase. No new male contraceptives have been introduced since the vasectomy in the mid-19th century. International and national studies have shown that over half of men would be willing to use new forms of male contraception, yet no new forms of male contraception exist on the market.

### Description of Related Art

Currently, several methods of male contraception are under investigation. These methods can be broken down into two main categories: hormonal and vas-occlusive. (Amory, J. K. Development of Novel Male Contraceptives. Clin. Transl. Sci. 13, 228-237, 2020). Hormonal options such as hormone pills and creams often cause side-effects similar to those observed in female hormonal contraceptives. Some side effects of hormonal male contraceptives include mood swings, depression, and increased risk of suicide. Vas-occlusion has a similar mechanism of action to a vasectomy in that the sperm are prevented from entering into the ejaculatory duct, with the key difference that vas-occlusion does not sever the vessel.

With the advent of novel male contraceptives including hormonal and non-hormonal methods, it is unclear how these male contraceptives will affect the rate of sexually transmitted infections (STIs). One area of interest in public health are multipurpose prevention technologies (MPT), which are combination products that simultaneously provide the user contraception while preventing the contraction of certain STIs. Currently, research on MPTs focus on female patients: for example, intravaginal rings that may deliver antiretroviral drugs for the prevention of HIV. The field lacks a male MPT that provides effective and reversible contraception for men while preventing the contraction of one or more STIs, which may otherwise be referred to as sexually transmitted disease or STDs.

The most publicized vas-occlusive technologies to date are reversible inhibition of sperm under guidance (RISUG) and VASALGEL (pre hydrolyzed RISUG). (Khilwani, B., Badar, A., Ansari, A. S. & Lohiya, N. K. RISUG® as a male contraceptive: journey from bench to bedside. Basic Clin. Androl. 30, (2020); Colagross-Schouten, A., Lemoy, M.-J., Keesler, R. I., Lissner, E. & VandeVoort, C. A. The contraceptive efficacy of intravas injection of VASALGEL for adult male rhesus monkeys. Basic Clin. Androl. 27, 4, 2017.) Both RISUG and VASALGEL are formulations of styrene maleic anhydride (SMA) dissolved in dimethyl sulfoxide (DMSO). Upon injection into the vas deferens (vas), SMA precipitates to coat the inner wall of the vas. The mechanism of action has been reported as both occlusive and spermicidal (depending on the formulation) with the spermicidal action believed to be due to the negative charge present after hydrolysis of the anhydride moieties (Guha, S. K. Biophysical mechanism-mediated time-dependent effect on sperm of human and monkey vas implanted polyelectrolyte contraceptive. Asian J. Androl. 9, 221-227 (2007); Waller, D., Bolick, D., Lissner, E., Premanandan, C. & Gamerman, G. Azoospermia in rabbits following an intravas injection of VASALGEL. Basic Clin. Androl. 26, 6, 2016.) Removal of the SMA from the vas is facilitated by flushing the vas with bicarbonate solution; however, in animal models the sperm lacked acrosomes post-reversal. (Waller, D., Bolick, D., Lissner, E., Premanandan, C. & Gamerman, G. Reversibility of VASALGEL male contraceptive in a rabbit model. Basic Clin. Androl. 27, 2017.)

Another area of interest are compositions and devices for testosterone replacement therapy (TRT). Currently, TRT involves the implantation of a pellet, which has an extrusion rate of 10% (see Shoskes J J, Wilson M K, Spinner M L. Pharmacology of testosterone replacement therapy preparations. Transl Androl Urol. 2016; 5(6):834-843, doi:10.21037/tau.2016.07.10). Other adverse events of TRT include site infections, bleeding, and fibrosis. The administration of the pellets also involves an invasive skin incision. This invasive procedure and adverse events may be completely circumvented with an injectable hydrogel approach.

Over the past several decades, hydrogels, which are water-swollen polymer networks, have been applied to a wide variety of biomedical applications including drug delivery. (Li, J. & Mooney, D. J. Designing hydrogels for controlled drug delivery. Nat. Rev. Mater. 1, 1-17, 2016.) Injectable hydrogels have been shown to be biocompatible with the ability to tune the mechanism of gelation, gelation rates, degradation mechanism, degradation rates, pore/mesh size, swelling ratio, and mechanical properties (Bakaic, E., Smeets, N. M. B. & Hoare, T. Injectable hydrogels based on poly(ethylene glycol) and derivatives as functional biomaterials, RSC Adv 5, 35469-35486, 2015; Avery, R. K. et al. An injectable shear-thinning biomaterial for endovascular embolization, Sci. Transl. Med. 8, 365ra156, 2016; Norouzi, M., Nazari, B. & Miller, D. W. Injectable hydrogel-based drug delivery systems for local cancer therapy, Drug Discov. Today 21, 1835-1849, 2016; Staruch, R. M. T., Glass, G. E., Rickard, R., Hettiaratchy, S. P. & Butler, P. E. M. Injectable Pore-Forming Hydrogel Scaffolds for Complex Wound Tissue Engineering: Designing and Controlling Their Porosity and Mechanical Properties, Tissue Eng. Part B Rev. 23, 183-198, 2017; Tan, H. & Marra, K. G. Injectable, Biodegradable Hydrogels for Tissue Engineering Applications, Materials 3, 1746-1767, 2010), thus making them an ideal candidate for the development of a vas-occlusive contraceptive.

Additional efforts in this area include those described in Chinese Patent CN1812746B and Japanese Patent JP5330533B2, which are hereby incorporated by reference herein in their entireties, but as with any art improvements are needed.

### SUMMARY OF THE INVENTION

Disclosed are compositions and methods of using one or more hydrogel for contraception as well as localized, sustained delivery of drugs. The hydrogels are easily injectable, have a quick gelation rate, are highly durable, and are able to last greater than 3 months in vivo, such as up to 3 months or more, including from up to 6 months, up to 1 year, or up to 5 years, or any time in between.

In particular embodiments, compositions are provided comprising: a first component and a second component capable of combination to form a hydrogel. Such compositions can in particular comprise a first component and a second component capable of combination to form a hydrogel, wherein, at a gelation time after the combination, the combination has a storage modulus (G′) and a loss modulus (G″), such that a ratio of G″ to G′ is less than about 1; and/or wherein the combination is capable of forming the hydrogel within a gelation rate of less than about 120 seconds.

Such compositions/hydrogels can be introduced to, into or on the body/patient any of numerous ways, including by way of the delivery devices described in U.S. Patent Application Publication No. 2020/0146876, entitled Systems and Methods for Delivering Biomaterials, which reference is hereby incorporated by reference in its entirety herein. Such delivery systems and methods include those capable of forming and delivering biomaterials from two components. Any apparatus or method for performing delivery of multicomponent delivery of biomaterials into or onto a body part, such as a body part, organ, duct, cavity/space or lumen, can be used. In some embodiments, the apparatus and methods are directed towards controlled delivery of micro-volumes of biomaterials into or onto a target location, the micro-volumes being defined as 0.001 mL-1 mL (or 1 μL-1,000 μL) of volume.

The hydrogels may be used for occlusion of a bodily duct, such as the vas deferens and/or fallopian tubes, for male and female contraception, respectively. In embodiments, once implanted, the hydrogel is capable of releasing one or more drugs, such as small molecules or biologics, indirectly, systemically, and/or directly to the site of interest over a sustained period of time. This is different than most combination products used for contraception such as intravaginal rings or the subcutaneous implants (i.e. IMPLANON, NEXPLANON) that are pre-formed devices. In embodiments, the hydrogel products described herein are injectable and can additionally be formed in situ, rather than pre-formed, which allows the user to control the drug delivery to a specific area of interest. In addition to acting as an occlusive agent, the hydrogels may also be used as a depot for drug delivery in the body, such as in the subcutaneous space, interstitial space, or for sealing, spraying, and/or embolization.

In addition to providing contraception and the prevention or treatment of one or more STIs, the hydrogels may be used to deliver other small molecules such as steroids. Such a product may be used, for example, for testosterone replacement therapy (TRT).

Aspects of the invention include Aspect 1, which is a method of delivering a therapeutic agent to a patient in need thereof, comprising: administering a device or hydrogel comprising the therapeutic agent to the patient; and allowing the device or hydrogel to release the therapeutic agent to the patient. In embodiments the device or hydrogel can be configured to degrade in part or in whole in response to one or more stimulus such that the device or hydrogel releases the drug to the patient, for example over a period of time. In embodiments of Aspect 1, the device or hydrogel is configured to have a lifetime that is as long or longer than the drug release profile.

Aspect 2 is the method of Aspect 1, wherein the administering of the device or hydrogel comprises injecting the hydrogel.

Aspect 3 is the method of Aspect 1 or 2, wherein the stimulus is an endogenous stimulus. In embodiments, the endogenous stimulus can be water or an aqueous environment.

Aspect 4 is the method of any preceding Aspect, wherein the stimulus is an exogenous stimulus. In embodiments, the exogenous stimulus can be light. In embodiments, the light can be ultraviolet, visible, or infrared light.

Aspect 5 is the method of any preceding Aspect, wherein the hydrogel comprises a stimulus-responsive molecule or moiety. In embodiments, the stimulus-responsive molecule or moiety can be photolabile.

Aspect 6 is the method of any preceding Aspect, wherein the administering of the hydrogel comprises intraluminal administration. In embodiments, the intraluminal administration can comprise occlusion of a body part, duct, organ, cavity/space or lumen.

Aspect 7 is the method of any preceding Aspect, wherein the body part, duct, organ, cavity/space or lumen is, or the administering/injecting involves injecting the composition/hydrogel into an artery such as a femoral artery, popliteal artery, coronary and/or carotid artery, esophagus, cavity, nasopharyngeal cavity, ear canal and tympanic cavity, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct such as a bile duct, a hepatic duct, a cystic duct, a pancreatic duct, or a parotid duct, organ such as a uterus, prostate, or any organ of the gastrointestinal tract or circulatory system or respiratory system or nervous system, or any urological organ.

Aspect 8 is the method of any preceding Aspect, wherein the administering of the device or hydrogel comprises administering two or more substances which are capable of forming a hydrogel before, during and/or after administration, such as before, during and/or after administration to the patient. In embodiments, the forming of the device or hydrogel can occur by way of a bioorthogonal reaction. In embodiments, the bioorthogonal reaction is Click chemistry.

Aspect 9 is the method of any preceding Aspect, wherein the therapeutic agent is a small molecule. In embodiments, the therapeutic agent can be a biologic. In embodiments, the therapeutic agent has pharmacological activity against one or more sexually transmitted diseases and/or is a male and/or female contraceptive. In embodiments, the therapeutic agent is doxycycline.

Aspect 10 is the method of any preceding Aspect, wherein the body part, duct, organ, cavity/space or lumen is a vas deferens or fallopian tube.

Aspect 11 is a hydrogel prepared by the method of any preceding Aspect.

Aspect 12 is a hydrogel comprising: one or more component; one or more therapeutic agent associated with the one or more component by way of one or more molecular bonds; wherein the hydrogel is capable of degrading upon exposure to a stimulus such that the hydrogel releases the therapeutic agent, for example over a period of time. In embodiments of Aspect 12, the hydrogel is configured to have a lifetime that is as long or longer than the drug release profile.

Aspect 13 is a hydrogel of any preceding Aspect or prepared by a method of any preceding Aspect, wherein the stimulus is an endogenous stimulus. In embodiments of Aspect 13, the endogenous stimulus is water or an aqueous environment. In further embodiments of Aspect 13, the stimulus is an exogenous stimulus. In embodiments, the exogenous stimulus is light. Still further, in embodiments of Aspect 13, the light is ultraviolet, visible, or infrared light.

Aspect 14 is a hydrogel of any preceding Aspect or prepared by a method of any preceding Aspect, wherein the hydrogel comprises a stimulus-responsive molecule or moiety. In embodiments of Aspect 14, the stimulus-responsive molecule or moiety is photolabile.

Aspect 15 is a hydrogel of any preceding Aspect or prepared by a method of any preceding Aspect, wherein the hydrogel is capable of being formed from two or more substances. In embodiments of Aspect 15, the hydrogel is capable of being formed by a bioorthogonal reaction. In further embodiments of aspect 15, the bioorthogonal reaction is Click chemistry.

Aspect 16 is a hydrogel of any preceding Aspect or prepared by a method of any preceding Aspect, wherein the one or more therapeutic/active/drug agent comprises a small molecule. In embodiments, one or more of the therapeutic/active/drug agents comprises a biologic. In embodiments of Aspect 16, one or more of the therapeutic/active/drug agents has pharmacological activity against one or more sexually transmitted diseases and/or provides for male and/or female contraception. In further embodiments of Aspect 16, the therapeutic agent is doxycycline, or any antibiotic.

Aspect 17 is a device comprising: a thermo-sensitive hydrogel loaded with one or more therapeutic agent, such as an antibiotic and/or a male or female contraceptive.

Aspect 18 is a device of any preceding Aspect or prepared by a method of any preceding Aspect, wherein the hydrogel is loaded with an effective amount of therapeutic/active/drug agent, such as an antibiotic alone or in combination with other active agents, to render the device capable of administering or releasing the antibiotic over or for a selected period of time, such as over a few minutes when subjected to a stimulus at any interval such as on command, once, multiple times a day, week, month or year, daily, weekly, and/or yearly.

Aspect 19 is a hydrogel loaded with antibiotic, such as a PEG hydrogel loaded with doxycycline, such as a hydrogel according to any preceding Aspect.

Aspect 20 is a device of any preceding Aspect or prepared by a method of any preceding Aspect, configured such that when deposited in subcutaneous space of a human or animal, the device serves as a drug depot.

Aspect 21 is a hydrogel loaded with testosterone, such as a PEG hydrogel loaded with testosterone, such as a hydrogel according to any preceding Aspect.

Aspect 22 is a hydrogel, such as a PEG hydrogel or a hydrogel according to any preceding Aspect, which hydrogel is loaded with emtricitabine and tenofovir disoproxil fumarate.

Aspect 23 is an in situ or injectable hydrogel system comprising a suspension of one or more hormone, such as a micronized hormone such as testosterone, such that upon injection the hormone or micronized testosterone is encapsulated within the hydrogel.

Aspect 24 is the hydrogel system of Aspect 23, wherein the hydrogel system comprises components capable of in situ gelling by way of bioorthogonal crosslinking for example of PEG hydrogel that encapsulates a suspension of one or more hormone such as micronized testosterone upon gelling, alone or in combination with other active agents.

Aspect 25 is the hydrogel system of Aspect 23 or 24, or a method of using the hydrogel system of Aspect 23 or 24, wherein the hydrogel system is configured to be placed or is placed into subcutaneous space of a human or animal and configured to deliver the hormone such as testosterone for up to 4 months and then begins to degrade or degrades, for example in a manner such that the size and properties of degradation products are capable of being excreted out. In embodiments of Aspect 25, the hydrogel is configured to have a lifetime that is as long or longer than the drug/hormone release profile.

Aspect 26 is an in situ or injectable hydrogel system comprising a suspension of one or more hormone, such as hormone or testosterone loaded polymeric microparticles alone or in combination with other active agents. In embodiments of Aspect 26, the hydrogel can be configured such that upon injection the hormone or testosterone loaded particles are encapsulated within the hydrogel. Such hydrogel systems can comprise components capable of in situ gelling by way of bioorthogonal crosslinking for example of PEG hydrogel wherein the hormone or testosterone is encapsulated in a polymeric microparticle that is entrapped within the hydrogel upon injection, such as upon injection into a subcutaneous space of a human or animal.

Aspect 27 is a hydrogel system of Aspect 26, wherein the hydrogel system is configured to deliver the hormone such as testosterone for up to 6 months and then begins to degrade or degrades, for example in a manner such that the size and properties of degradation products are capable of being excreted out. In embodiments of Aspect 27, the hydrogel system is configured to have a lifetime that is as long or longer than the drug/testosterone release profile.

Aspect 28 is an in situ or injectable hydrogel that is loaded with a suspension of anti-HIV drugs/anti-virals or drug delivery vehicles (such as those listed above) loaded with the drug/antivirals, for example, an in situ gelling via bioorthogonal crosslinking of PEG hydrogel loaded with emtricitabine and tenofovir disoproxil fumarate that is capable of being implanted or that is implanted within the vasa deferentia, whereby the hydrogel for example is capable of occluding the vessel resulting in infertility and/or prevention of HIV infection for up to the lifetime of the implant, wherein for example the lifetime of the implant is up to 6 months, 12 months, 18 months, or 24 months.

Aspect 29 is an in situ or injectable hydrogel that is loaded with a suspension of anti-HIV drugs/anti-virals or drug delivery vehicles (such as those listed above) loaded with the drugs/antivirals, for example, an in situ gelling via bioorthogonal crosslinking of PEG hydrogel loaded with emtricitabine and tenofovir disoproxil fumarate that is capable of being implanted or that is implanted within one or more fallopian tubes, whereby the hydrogel for example is capable of occluding the tubes resulting in infertility and/or prevention of HIV infection for up to the lifetime of the implant, wherein for example the lifetime of the implant is up to 6 months, 12 months, 18 months, or 24 months.

Aspect 30 is an in situ or injectable hydrogel that is loaded with a suspension of antibiotics/anti-virals to prevent one or more sexually transmitted infections or diseases or that is loaded with drug delivery vehicles (such as those listed above) loaded with the drugs/antivirals, for example, an in situ gelling via bioorthogonal crosslinking of PEG hydrogel loaded with one or more antibiotic, such as doxycycline e.g., soluble doxycycline that is implanted or capable of being implanted within a vessel of a human or animal, such as implanted or capable of being implanted in the vasa deferentia, whereby the hydrogel is capable of occluding the vessel resulting in infertility and/or prevention of one or more sexually transmitted disease or infection, such as chlamydia, gonorrhea, and/or syphilis infection for up to the lifetime of the implant, wherein for example the lifetime of the implant is up to 6 months, 12 months, 18 months, or 24 months.

Aspect 31 is an in situ or injectable hydrogel that is loaded with a suspension of antibiotics/anti-virals to prevent sexually transmitted infections or drug delivery vehicles (such as those listed above) loaded with the drugs/antivirals, for example, an in situ gelling via bioorthogonal crosslinking of PEG hydrogel loaded with soluble doxycycline that is implanted within the fallopian tubes, wherein the hydrogel occludes or is capable of occluding the tubes resulting in infertility and/or prevention of chlamydia, gonorrhea, and/or syphilis infection for up to the lifetime of the implant, wherein for example the lifetime of the implant is up to 6 months, 12 months, 18 months, or 24 months.

Aspect 32 is an in situ or injectable hydrogel that is loaded with a suspension of antibiotics/anti-virals to prevent sexually transmitted infections or drug delivery vehicles (such as those listed above) loaded with the drugs/antivirals, for example, an in situ gelling via bioorthogonal crosslinking of PEG hydrogel loaded with a suspension of nanocrystals of doxycycline that is capable of being implanted or that is implanted within the vasa deferentia, whereby the hydrogel occludes or is capable of occluding the vessel resulting in infertility and/or prevention of chlamydia, gonorrhea, and/or syphilis infection, wherein for example the lifetime of the implant is up to 6 months, 12 months, 18 months, or 24 months.

Aspect 33 is an in situ or injectable hydrogel that is loaded with a suspension of antibiotics/anti-virals to prevent one or more sexually transmitted infections or drug delivery vehicles (such as those listed above) loaded with the drugs/antivirals, for example, an in situ gelling via bioorthogonal crosslinking of PEG hydrogel loaded with a suspension of nanocrystals of doxycycline that is implanted or capable of being implanted within the fallopian tubes, whereby the hydrogel occludes or is capable of occluding the tubes resulting in infertility and/or prevention of chlamydia, gonorrhea, and/or syphilis infection for up to the lifetime of the implant, wherein for example the lifetime of the implant is up to 6 months, 12 months, 18 months, or 24 months.

Aspect 34 is a composition, comprising: a first component and a second component, the first component formulated to be crosslinked with the second component to form a hydrogel; the first component and the second component being formulated to have an initial storage modulus (initial G′) and an initial loss modulus (initial G″) when the first component and the second component are initially combined, a ratio of the initial G″ to the initial G′ being between about 5 and about 100; and the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds.

Aspect 35 is the composition of Aspect 34, wherein the gelation time is less than about 60 seconds, less than about 30 seconds, less than about 10 seconds and/or immediate.

Aspect 36 is the composition of Aspect 34 or 35, wherein: the first component is a PEG based component, such as a multi-arm polyethylene glycol, optionally terminated with thiol; and the second component is a PEG based component, such as a multi-arm polyethylene glycol, optionally terminated with a maleimide.

Aspect 37 is the composition of any of Aspects 34-36, wherein at least one of the first component and/or the second component are dissolved in a solvent and have a weight percentage within the solvent of between about 1 wt % and 40 wt %, such as up to 25 wt %, or up to 30 wt %, e.g., between about 1 wt % and 20 wt %, up to about 20 wt %, or between about 5 and 15 wt %.

Aspect 38 is the composition of Aspect 37, wherein the solvent is any one or more of: Acetic Acid-Sodium Acetate (AA), Citric Acid-Sodium Citrate (CA), Citric Acid (0.2 M)-Phosphate Buffer (0.1 M) (CP), or Phosphate Buffer (PB).

Aspect 39 is the composition of Aspect 38, wherein the first component comprises any one or more functional groups chosen from Thiol (SH), Maleimide (MAL), nitrobenzyl (e.g., o-nitrobenzyl, ONB), Hydrazide (HZ), Isocyanate (IC), Amine (NH), Succinimidyl Glutaraldehyde (SG), Aldehyde (AD), or Epoxide (EP) and/or wherein the second component comprises any one or more of Thiol (SH), Maleimide (MAL), nitrobenzyl (e.g., o-nitrobenzyl, ONB), Hydrazide (HZ), Isocyanate (IC), Amine (NH), Succinimidyl Glutaraldehyde (SG), Aldehyde (AD), or Epoxide (EP).

Aspect 40 is a delivery system, comprising: a container assembly containing a first component and a second component, the first component being separate from the second component within the container assembly, the first component formulated to be crosslinked with the second component to form a hydrogel, the first component and the second component being formulated such that the hydrogel has a gelation time; a connector configured to be coupled to the container assembly; a delivery member configured to be coupled to the connector, the delivery member configured to be inserted into a body part, such as an organ, duct, cavity/space or a lumen; and a drive assembly configured to be operatively coupled to the container assembly, the drive assembly configured to move a first plunger within the first container to convey a portion of the first component from the first container and a second plunger within the second container to convey a portion of the second component from the second container, the drive assembly configured to move the first plunger and the second plunger to convey a portion of the first component and a portion of the second component through the connector and out of the delivery member within a delivery time that is less than the gelation time. In embodiments of Aspect 40, the delivery member is configured to be inserted into a body part, duct, organ, cavity/space or lumen chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

Aspect 41 is the delivery system of Aspect 40, wherein the first component and the second component are formulated to have an initial storage modulus (initial G′) and an initial loss modulus (initial G″) when the first component and the second component are initially combined, a ratio of the initial G″ to the initial G′ being between about 5 and about 100; and the first component and the second component are formulated to have a delivered storage modulus (delivered G′) and a delivered loss modulus (delivered G″) when the first component and the second component are conveyed out of the delivery member, a ratio of the delivered G″ to the delivered G′ being between about 1/3 and about 3. In some embodiments of Aspect 41, the ratio of the initial G″ to the initial G′ is between about 30 and about 50; and the ratio of the delivered G″ to the delivered G′ is between about 1/3 and about 1.

Aspect 42 is the delivery system of Aspect 41 or 40, wherein the first component is a PEG based component, such as a multi-arm polyethylene glycol, optionally terminated with thiol; and the second component is a PEG based component, such as a multi-arm polyethylene glycol, optionally terminated with a maleimide.

Aspect 43 is the delivery system of any of Aspects 40-42, wherein at least one of the first component and/or the second component includes a therapeutic agent.

Aspect 44 is the delivery system of Aspect 43, wherein the therapeutic agent includes at least one of a small molecule, a biologic, an antibiotic and/or an anti-viral.

Aspect 45 is the delivery system of any of Aspects 40-44, wherein at least one of the first component and/or second component are dissolved in a solvent chosen from one or more of Acetic Acid-Sodium Acetate (AA), Citric Acid-Sodium Citrate (CA), Citric Acid (0.2 M)-Phosphate Buffer (0.1 M) (CP), or Phosphate Buffer (PB); and have a weight percentage within the solvent of between about 1 wt % and 30 wt %, or combinations thereof.

Aspect 46 is the delivery system of any of Aspects 40-45, wherein the portion of the first component and the portion of the second component produce a delivered volume between about 50 microliters and about 250 microliters.

Aspect 47 is the delivery system of any of Aspects 40-46, wherein the delivery member is any one of a catheter or a needle.

Aspect 48 is the delivery system of any of Aspects 40-47, wherein the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds.

Aspect 49 is a method of delivering a composition, comprising: coupling a container assembly to a delivery member, the container assembly defining a first chamber and a second chamber, the first chamber being fluidically isolated from the second chamber and containing a first component and the second chamber containing a second component, the first component formulated to be crosslinked with the second component to form a hydrogel, the first component and the second component being formulated such that the hydrogel has a gelation time; and conveying a portion of the first component and a portion of the second component into a mixing volume of the delivery member and through the delivery member within a delivery time that is less than the gelation time, the first component crosslinking with the second component to at least partially form the hydrogel within the delivery member such that the conveying causes the hydrogel to be conveyed out of an exit opening of the delivery member.

Aspect 50 is the method of Aspect 49, wherein the first component and the second component are formulated such that a viscoelastic substance is conveyed out of the exit opening of the delivery member.

Aspect 51 is the method of Aspect 49 or 50, wherein the first component and the second component are formulated to have an initial storage modulus (initial G′) and an initial loss modulus (initial G″) when the first component and the second component are initially combined, a ratio of the initial G″ to the initial G′ being between about 5 and about 100; and the first component and the second component are formulated to have a delivered storage modulus (delivered G′) and a delivered loss modulus (delivered G″) when the first component and the second component are conveyed out of the delivery member, a ratio of the delivered G″ to the delivered G′ being between about 1/3 and about 3. In some embodiments, the ratio of the initial G″ to the initial G′ is between about 30 and about 50; and the ratio of the delivered G″ to the delivered G′ is between about 1/3 and about 1.

Aspect 52 is the method of any of Aspects 49-51, wherein the conveying of the hydrogel out of the delivery member includes conveying the hydrogel to a body part, such as an organ, duct, cavity/space or a body lumen.

Aspect 53 is the method of Aspect 52, wherein the wherein the body part is chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

Aspect 54 is the method of Aspect 52 or 53, wherein the hydrogel conveyed to the body part, organ, duct, cavity/space or lumen at least partially occludes the body part, duct, organ, cavity/space or lumen.

Aspect 55 is the method of any of Aspects 49-54, wherein the first component is a PEG based component, such as a multi-arm polyethylene glycol, optionally terminated with thiol; and the second component is a PEG based component, such as a multi-arm polyethylene glycol, optionally terminated with a maleimide.

Aspect 56 is the method of any of Aspects 49-55, wherein at least one of the first component and/or the second component includes a therapeutic agent.

Aspect 57 is the method of Aspect 56, wherein the therapeutic agent includes at least one of a small molecule, a biologic, an antibiotic and/or an anti-viral.

Aspect 58 is a kit comprising: a first container containing a first component, the first component being a PEG based component, such as a multi-arm polyethylene glycol, optionally terminated with thiol; a second container containing a second component, the second component being a PEG based component, such as a multi-arm polyethylene glycol, optionally terminated with a maleimide; and a delivery device. The delivery device includes: a container assembly configured to receive the first container and the second container; a connector configured to be coupled to the container assembly; and a drive assembly configured to move a first plunger within the first container to convey a portion of the first component from the first container and to move a second plunger within the second container to convey a portion of the second component from the second container, the drive assembly configured to move the first plunger and the second plunger to convey a portion of the first component and a portion of the second component through the connector.

Aspect 59 is the kit of Aspect 58, wherein at least one of the first component and/or the second component includes a therapeutic agent.

Aspect 60 is the kit of Aspect 58 or 59, wherein the therapeutic agent includes at least one of a small molecule, a biologic, an antibiotic and/or an anti-viral.

Aspect 61 is the kit of any of Aspects 58-60, wherein the kit further comprises a delivery member configured to be coupled to the connector, the delivery member configured to be inserted into a body part, such as an organ, duct, cavity/space or lumen; and the drive assembly is configured to move the first plunger and the second plunger to convey the portion of the first component and the portion of the second component through the connector and out of the delivery member within a delivery time.

Aspect 62 is the kit of Aspect 61, wherein the body part, organ, duct, cavity/space or lumen is chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

Additional Aspects include Aspect 63, which is a composition, comprising: a first component and a second component, the first component formulated to be crosslinked with the second component to form a hydrogel; the first component and the second component being formulated to have an initial storage modulus (initial G′) and an initial loss modulus (initial G″) when the first component and the second component are initially combined, a ratio of the initial G″ to the initial G′ being between about 5 and about 100; and the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds; optionally further comprising one or more therapeutic agent.

In embodiments of Aspect 63, the first component and/or second component may be one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In further embodiments of Aspect 63, the therapeutic agent (also referred to herein as active agent or drug) can be added to one or more of the substances/components (such as either or both of the first and second components) that cross-link to form the hydrogel. For example, as an embodiment of Aspect 63, if a hydrogel is formed from two macromers, the drug can be loaded to one of the macromers while in solution, while the other macromer does not contain any drug(s), or contains the same drug/therapeutic/active agent, or another drug/therapeutic/active agent. In still further embodiments of Aspect 63, the drug/therapeutic/active agent(s) may be loaded in the same or varying concentrations in the components/constituents used to form the hydrogel.

Aspect 64 is the composition of Aspect 63, wherein: the hydrogel comprises the therapeutic agent(s); and the hydrogel is configured to release the therapeutic agent(s) to a patient, for example, over a period of at least 5 days, or at least a week, or at least a month.

Aspect 65 is the composition of Aspect 63 or 64, wherein the therapeutic agent(s) are chosen from one or more of hormones, anti-HIV drugs, antibiotics, and/or anti-virals.

Aspect 66 is the composition of any of Aspects 63-65, wherein the gelation time is less than about 60 seconds.

Aspect 67 is the composition of any of Aspects 63-66, wherein: the first component is a PEG based component, such as a multi-arm polyethylene glycol, optionally terminated with thiol; and/or the second component is a PEG based component, such as a multi-arm polyethylene glycol, optionally terminated with a maleimide.

Aspect 68 is the composition of any of Aspects 63-67, wherein the hydrogel is capable of being disposed within one or more body part, organ, duct, cavity/space or lumen chosen from: an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

Aspect 69 is the composition of any of Aspects 63-68, wherein the hydrogel is capable of occluding the body part, organ, duct, cavity/space or lumen, in whole or in part, in a manner to: cause infertility; and/or prevent or treat one or more infection or disease, such as a sexually transmitted infection or disease or HIV infection, for up to 1 week, 1 month, 3 months, 6 months, 12 months, 18 months, or 24 months.

Aspect 70 is the composition of any of Aspects 63-69, wherein at least one of the first component and/or the second component are dissolved in a solvent and have a weight percentage within the solvent of between about 1 wt % and 30 wt %, such as between about 1-25 wt %.

Aspect 71 is the composition of any of Aspects 63-70, wherein at least one of the first component and/or the second component are dissolved in a solvent chosen from any one or more of: Acetic Acid-Sodium Acetate (AA), Citric Acid-Sodium Citrate (CA), Citric Acid (0.2 M)-Phosphate Buffer (0.1 M) (CP), or Phosphate Buffer (PB).

Aspect 72 is the composition of any of Aspects 63-71, wherein the first and/or second component comprises any one or more functional group chosen from Thiol (SH), Maleimide (MAL), o-nitrobenzyl (e.g., o-nitrobenzyl, ONB), Hydrazide (HZ), Isocyanate (IC), Amine (NH), Succinimidyl Glutaraldehyde (SG), Aldehyde (AD), or Epoxide (EP).

Additional embodiments of Aspects 63-72 include use of the composition in providing male or female contraception and/or prophylaxis against one or more sexually transmitted infections or diseases; and/or manufacture of a medicament comprising any one or more composition of Aspects 63-72 for providing infertility to a male or female subject and/or for treating one or more sexually transmitted infections or diseases.

Aspect 73 is a hydrogel comprising: one or more component; and one or more therapeutic agent (also referred to as a drug or active agent) associated with the one or more component by way of one or more molecular bonds; wherein the hydrogel is capable of releasing the therapeutic agent over a period of time, for example, over a period of at least 5 days, or at least a week, or at least a month.

In embodiments of Aspect 73, the hydrogel may be formed from and/or comprise one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In further embodiments of Aspect 73, the therapeutic agent (also referred to herein as active agent or drug) can be added to one or more substances/components that may be used to cross-link to form the hydrogel. For example, as an embodiment of Aspect 73, if a hydrogel is formed from two macromers, the drug can be loaded to one of the macromers while in solution, while the other macromer does not contain any drug(s), or contains the same drug/therapeutic/active agent, or another drug/therapeutic/active agent. In still further embodiments of Aspect 73, the drug/therapeutic/active agent(s) may be loaded in the same or varying concentrations in the components/constituents used to form the hydrogel.

Aspect 74 is the hydrogel of Aspect 73, wherein the hydrogel is capable of being formed by a bioorthogonal reaction.

Aspect 75 is the hydrogel of Aspect 73 or 74, wherein one or more of the therapeutic agents has pharmacological activity against one or more sexually transmitted diseases and/or provides for male and/or female contraception.

Aspect 76 is the hydrogel of any of Aspects 73-75, wherein one or more of the components comprises: a first component and a second component, the first component formulated to be crosslinked with the second component to form the hydrogel; the first component and the second component being formulated to have an initial storage modulus (initial G′) and an initial loss modulus (initial G″) when the first component and the second component are initially combined, a ratio of the initial G″ to the initial G′ being between about 5 and about 100; and the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds.

Aspect 77 is the hydrogel of any of Aspects 73-76, wherein: the first component is a PEG based component, such as a multi-arm polyethylene glycol, optionally terminated with thiol; and the second component is a PEG based component, such as a multi-arm polyethylene glycol, optionally terminated with a maleimide.

Aspect 78 is the hydrogel of any of Aspects 73-77, wherein: the first component comprises one or more of the therapeutic agents (also referred to as an active agent and/or drug); or the second component comprises one or more of the therapeutic agents; or the first and second components comprise one or more of the therapeutic agents; or the first component comprises a first therapeutic agent and the second component comprises a second therapeutic agent that is the same or different as the first therapeutic agent.

Aspect 79 is the hydrogel of any of Aspects 73-78, wherein: at least one of the first and/or second component are dissolved in a solvent chosen from one or more of Acetic Acid—Sodium Acetate (AA), Citric Acid-Sodium Citrate (CA), Citric Acid (0.2 M)-Phosphate Buffer (0.1 M) (CP), or Phosphate Buffer (PB); at least one of the first and/or second component and have a weight percentage within the solvent of between about 1 wt % and 30 wt %; and at least one or the first and/or second component comprises any one or more functional group chosen from Thiol (SH), Maleimide (MAL), nitro-benzyl (e.g., o-nitrobenzyl, ONB), Hydrazide (HZ), Isocyanate (IC), Amine (NH), Succinimidyl Glutaraldehyde (SG), Aldehyde (AD), or Epoxide (EP).

Additional embodiments of Aspects 73-79 include use of the hydrogel in providing male or female contraception and/or prophylaxis against one or more sexually transmitted infections or diseases; and/or manufacture of a medicament comprising any one or more hydrogel of Aspects 73-79 for providing infertility to a male or female subject and/or for treating one or more sexually transmitted infections or diseases.

Aspect 80 is a composition comprising: a first component and a second component, the first component formulated to be crosslinked with the second component to form a hydrogel; wherein the first and/or second component comprise a polyethylene glycol based component comprising one or more maleimide functional group; wherein (i) the first component comprises one or more therapeutic agents; or (ii) the second component comprises one or more therapeutic agents; or (iii) the first and second components comprise one or more therapeutic agents; or (iv) the first component comprises a first therapeutic agent and the second component comprises a second therapeutic agent that is the same or different as the first therapeutic agent; and optionally wherein the hydrogel is capable of releasing the therapeutic agent(s) to the patient, for example, over a period of at least 5 days, or at least a week, or at least a month.

Aspect 81 is an in situ or injectable hydrogel comprising: one or more hormone, anti-HIV drug, antibiotic, and/or anti-viral (also referred to as a therapeutic agent, active agent and/or drug); wherein the hydrogel is configured to be disposed in a body part, organ, duct, cavity/space or lumen chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space; and wherein the hydrogel is capable of occluding the body part, organ, duct, cavity/space or lumen, in whole or in part, in a manner to cause infertility and/or prevent or treat one or more infection or disease, such as a sexually transmitted infection or disease or HIV infection, for up to 1 week, 1 month, 3 months, 6 months, 12 months, 18 months, or 24 months.

In embodiments of Aspects 80 and/or 81, the composition or hydrogel may comprise or be formed from one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In further embodiments of Aspects 80 and 81, the therapeutic agent (also referred to herein as active agent or drug) can be added to one or more substances/components that may be used to cross-link to form the composition/hydrogel. For example, as an embodiment of Aspects 80 and 81, if a composition/hydrogel is formed from two macromers, the drug can be loaded to one of the macromers while in solution, while the other macromer does not contain any drug(s), or contains the same drug/therapeutic/active agent, or another drug/therapeutic/active agent. In still further embodiments of Aspects 80 and 81, the drug/therapeutic/active agent(s) may be loaded in the same or varying concentrations in the components/constituents used to form the hydrogel.

Aspect 82 is the composition/hydrogel of Aspect 81, wherein: the hydrogel is an in situ hydrogel comprising: a first component and a second component, the first component formulated to be crosslinked with the second component to form the hydrogel; wherein the first and/or second component comprise a polyethylene glycol based component comprising one or more maleimide functional group.

Additional embodiments of Aspects 80-82 include use of the composition/hydrogel in providing male or female contraception and/or prophylaxis against one or more sexually transmitted infections or diseases; and/or manufacture of a medicament comprising any one or more composition of Aspects 80-82 for providing infertility to a male or female subject and/or for treating one or more sexually transmitted infections or diseases.

Aspect 83 is a method comprising: delivering, injecting, administering, providing, conveying, placing, depositing or disposing a hydrogel comprising one or more therapeutic agent to a patient; wherein the hydrogel is capable of releasing the therapeutic agent to the patient, for example, over a period of at least 5 days, or at least a week, or at least a month.

In embodiments of Aspect 83, the hydrogel may comprise or may be formed from one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In further embodiments of Aspect 83, the therapeutic agent (also referred to herein as active agent or drug) can be added to one or more substances/components that may be used to cross-link to form the hydrogel. For example, as an embodiment of Aspect 83, if a hydrogel is formed from two macromers, the drug can be loaded to one of the macromers while in solution, while the other macromer does not contain any drug(s), or contains the same drug/therapeutic/active agent, or another drug/therapeutic/active agent. In still further embodiments of Aspect 83, the drug/therapeutic/active agent(s) may be loaded in the same or varying concentrations in the components/constituents used to form the hydrogel.

Aspect 84 is the method of Aspect 83, wherein the hydrogel is configured to degrade in part or in whole. In embodiments of Aspect 84, the hydrogel is configured to have a lifetime that is as long or longer than the drug/therapeutic agent/active agent release profile.

Aspect 85 is the method of Aspect 83 or 84, wherein the delivering, injecting, administering, providing, conveying, or disposing comprises injecting: a first component and a second component, the first component formulated to be crosslinked with the second component to form the hydrogel; the first component and the second component being formulated to have an initial storage modulus (initial G′) and an initial loss modulus (initial G″) when the first component and the second component are initially combined, a ratio of the initial G″ to the initial G′ being between about 5 and about 100; and the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds.

Aspect 86 is the method of Aspect 85, wherein: the first component is a PEG based component, such as a multi-arm polyethylene glycol, optionally terminated with thiol; and the second component is a PEG based component, such as a multi-arm polyethylene glycol, optionally terminated with a maleimide.

Aspect 87 is the method of Aspect 85 or 86, wherein at least one of the first and/or second component: are dissolved in a solvent chosen from one or more of Acetic Acid-Sodium Acetate (AA), Citric Acid-Sodium Citrate (CA), Citric Acid (0.2 M)-Phosphate Buffer (0.1 M) (CP), or Phosphate Buffer (PB); and have a weight percentage within the solvent of between about 1 wt % and 30 wt %.

Aspect 88 is the method of any of Aspects 83-87, wherein the delivering, injecting, administering, providing, conveying, placing, depositing or disposing comprises injecting, administering, providing, conveying, placing, depositing or disposing one or more component and/or the hydrogel into a body part, organ, duct, cavity/space or lumen chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

Aspect 89 is the method of any of Aspects 83-88, wherein the delivering, injecting, administering, providing, conveying, or disposing comprises injecting, administering, providing, conveying, placing, depositing or disposing one or more component and/or the hydrogel into a bladder, ureter, ejaculatory duct, epididymis, vas deferens, urethra, uterine cavity, vaginal canal, fallopian tube, cervix, a uterus, prostate, kidney, or any urological organ.

Aspect 90 is the method of any of Aspects 83-89, wherein the therapeutic agent has pharmacological activity against one or more sexually transmitted diseases and/or is a male and/or female contraceptive.

Aspect 91 is a method of providing contraception and prophylaxis, comprising: implanting a hydrogel into the vas deferens of a male subject; wherein the hydrogel is configured to provide male contraception; and wherein the hydrogel is configured to provide prophylaxis against one or more sexually transmitted diseases or infections.

In embodiments of Aspect 91, the hydrogel may comprise or be formed from one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In further embodiments of Aspect 91, the prophylaxis can be provided by one or more therapeutic agent (also referred to herein as active agent or drug), and can be added to one or more substances/components that may be used to cross-link to form the hydrogel. For example, as an embodiment of Aspect 91, if a hydrogel is formed from two macromers, the drug can be loaded to one of the macromers while in solution, while the other macromer does not contain any drug(s), or contains the same drug/therapeutic/active agent, or another drug/therapeutic/active agent. In still further embodiments of Aspect 91, the drug/therapeutic/active agent(s) may be loaded in the same or varying concentrations in the components/constituents used to form the hydrogel.

Aspect 92 is the method of Aspect 91, wherein the hydrogel comprises: a first component and a second component, the first component formulated to be crosslinked with the second component to form the hydrogel; wherein the first and/or second component comprise a polyethylene glycol based component, optionally one or both of the first and/or second component comprising one or more maleimide functional group.

### BRIEF DESCRIPTION OF THE DRAWINGS

The accompanying drawings illustrate certain aspects of embodiments of the present invention and should not be used to limit the invention. Together with the written description, the drawings serve to explain certain principles of the invention.

FIG. 1 is a graph showing the cumulative release percentage over time according to an embodiment of the invention in which a hydrogel is loaded with free doxycycline, wherein the cumulative release is 50.96% at 72 hours.

FIG. 2 is a graph showing the cumulative release percentage over time according to an embodiment of the invention in which a hydrogel is loaded with free doxycycline, wherein the cumulative release is 50.43% at 15 days.

FIG. 3 is a schematic of a portion of a delivery system according to an embodiment.

FIG. 4 is a schematic of a portion of a delivery system according to an embodiment.

FIG. 5 is schematic illustration of a portion of the delivery system shown in FIG. 3 and FIG. 4 inserted into a body part, duct, organ, cavity/space or lumen according to an embodiment.

FIG. 6 is a schematic illustration of the delivery system of FIG. 5 delivering biomaterial components to the body part, duct, organ, cavity/space or lumen.

FIG. 7 is a schematic illustration of the biomaterial components placed within the body part, organ, duct, cavity/space or lumen after the delivery system of FIG. 5 has been withdrawn from the body part, organ, duct, cavity/space or lumen.

FIG. 8 is a schematic diagram showing an insertion of a delivery member into a body part, organ, duct, cavity/space or lumen according to an embodiment.

FIG. 9 is a schematic diagram showing a coupling of the delivery member in FIG. 8 to a connector attached to a container assembly according to an embodiment.

FIG. 10 is a schematic diagram showing delivery of biomaterial components to the body part, organ, duct, cavity/space or lumen via the connector and delivery member of FIG. 9.

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Generally, embodiments of the invention relate to methods of administering a device or hydrogel comprising one or more active agent (e.g., a therapeutic agent) to the patient; and allowing the device or hydrogel to release the therapeutic agent to the patient.

The device or hydrogel can be formulated for contraception as well as for the delivery of drugs, such as the localized, sustained delivery of drugs over a desired period of time. In embodiments, the hydrogels are easily injectable, have a quick gelation rate, are highly durable, and are able to last greater than 3 months in vivo, such as up to 3 months or more, including from up to 6 months, up to 1 year, or up to 5 years, or any time in between.

In particular embodiments, compositions are provided comprising: a first component and a second component capable of combination to form a hydrogel. Such compositions can in particular comprise a first component and a second component capable of combination to form a hydrogel, wherein, at a gelation time after the combination, the combination has a storage modulus (G′) and a loss modulus (G″), such that a ratio of G″ to G′ is less than about 1; and/or wherein the combination is capable of forming the hydrogel within a gelation rate of less than about 120 seconds.

As used herein, the term “component” (also referred to as “biomaterial component”) includes any substance that is capable of forming a hydrogel and/or drug delivery device according to the invention, such as a biomaterial product. For example, a component can include a small molecule, catalyst, peptide, protein, enzyme, nucleotide (or derivatives of), short chains of nucleotides (or derivatives of), long chains of nucleotides (or derivatives of), monosaccharides (or derivatives of), disaccharides (or derivatives of), trisaccharides (or derivatives of), oligo saccharides (or derivatives of), polysaccharides (or derivatives of), monomer, oligomer, macromer, or polymer that can be cross-linked with another component to form a hydrogel and/or drug delivery device according to the invention (e.g., a delivered product or biomaterial product). A component can include a mixture or solution of one or more constituents (e.g., a polymer and a solvent). A component can include such constituents regardless of their state of matter (e.g., solid, liquid or gas). A component can include both active constituents and inert constituents. A constituent may be one or more of a therapeutic agent, an active agent, or drug. For example, in some embodiments, a component can include certain polymers that can form a delivered product, as well as a medicament or other active ingredient. By way of another example, in some embodiments a component can include drugs, including but not limited to, small molecule drugs and biologics. In other embodiments, a component can include certain constituents to impart desired properties to the delivered product, including constituents that facilitate the delivered product being echogenic, radiopaque, radiolucent, or the like.

In embodiments, the components (e.g., monomers, macromers, or polymers) that form the hydrogel have varied molecular weights, component ratios, concentrations/weight percentages of the components in solvent, and composition of the solvent. Varying any, some, or all of these properties can affect the mechanical, chemical, or biological properties of the device. This includes properties such as, but not limited to, dissolution time, gelation rate/time, porosity, biocompatibility, hardness, elasticity, viscosity, swelling, fluid absorbance, melting temperature, degradation rate, density, reversal time, and echogenicity. Accordingly, one of skill in the art based on this disclosure will know how to “tune” the particular desired features of a hydrogel to achieve a particular purpose and/or function for a particular application.

In embodiments, the hydrogel can be formed by having one or more substances/components/constituents cross-link with one or more of each other, such as macromers. In embodiments, the hydrogel can be formed in situ and/or otherwise at the time of insertion/injection and/or thereafter, such as immediately upon combination of components.

The hydrogel or its macromers can include components including, but not limited to, a polymer backbone, stimuli-responsive functional group(s), and functional groups that enable cross-linking. The functional groups that enable cross-linking can be end groups on the macromer(s). The cross-linking of the macromers may be via biorthogonal chemistry, such as a Click reaction. In one embodiment, a bioorthogonal reaction is utilized because it is highly efficient, has a quick gelation rate, occurs under mild conditions, and does not require a catalyst.

One example of such reaction is maleimide and thiol. Another type of Click reaction is cycloaddition, which can include a 1,3-dipolar cycloaddition or hetero-Diels-Alder cycloaddition or azide-alkyne cycloaddition, for example. The reaction can be a nucleophilic ring-opening. This includes openings of strained heterocyclic electrophiles including, but not limited to, aziridines, epoxides, cyclic sulfates, aziridinium ions, and episulfonium ions. The reaction can involve carbonyl chemistry of the non-aldol type including, but not limited to, the formation of ureas, thioureas, hydrazones, oxime ethers, amides, and aromatic heterocycles. The reaction can involve carbonyl chemistry of the aldol type. The reaction can also involve forming carbon-carbon multiple bonds, epoxidations, aziridinations, dihydroxylations, sulfenyl halide additions, nitrosyl halide additions, and Michael additions.

Another example of bioorthogonal chemistry is nitrone dipole cycloaddition. The Click chemistry can include a norbornene cycloaddition, an oxanobornadiene cycloaddition, a tetrazine ligation, a [4+1] cycloaddition, a tetrazole chemistry, or a quadricyclane ligation. Other end-groups include, but are not limited to, acrylic, cyrene, amino acids, amine, or acetyl. In one aspect, the end groups may enable a reaction between the polymeric device and the cells lining the tube, duct, tissue, or organ that is being occluded. For example, the devices, compositions, hydrogels and methods of the present invention can include any device, composition, method, hydrogel and/or component/constituent thereof disclosed in any one or more of U.S. Patent Application Publication Nos. 2017/0136143, 2017/0136144, US2018/0028715, US2018/0185096, US2019/0038454, US2019/0053790, US2019/0060513, WO2017/083753, WO2018/139369, WO2019/070632, U.S. Pat. No. 10,155,063, which are each incorporated by reference herein in their entireties.

The macromers or polymers that form hydrogels according to embodiments of the invention may be one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (LDPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or pol(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan; poly (methyl methacrylate), poly (vinyl alcohol), poly (urethanes) poly (ethylene) poly (siloxanes) or silicones, poly (vinyl pyrrolidone), poly (ethylene-co-vinyl acetate), poly (methyl methacrylate), poly (vinyl alcohol), poly (N-vinyl pyrrolidone), poly (acrylic acid), poly (2hydroxy ethyl methacrylate), polyacrylamide, poly (methacrylic glycol), poly (ethylene glycol), polyorthoesters, poly (lactide-co-glycolides) (PLGA), polyactide (PLA), polyanhydride, polyglycolides (PGA); polymers formed from radical polymerization such as polystyrene, poly(acrylic acid), poly(methacrylic acid), poly(ethyl methacrylate), poly(methyl methacrylate), poly(vinyl acetate), poly(ethyleneterepthalate), polyethylene, polypropylene, polybutadiene, polyacrylonitrile, poly(vinyl chloride), poly(vinylidene chloride), poly(vinyl alcohol), polychloroprene, polyisoprene, vinyl fluoride, vinylidene fluoride, trifluoroethylene, poly(methyl-α-chloracrylate), poly(ethylvinyl ketone), polymethacroleine, polyaurylmethacryate, poly(2-hydroxyethylmethamilate), poly(fumaronitrile), polychlorotrifluoroethylene, poly(acrylonitrile), polyacroleine, polyacenaphthylene, and branched polyethylene; natural polymers including silk, rubber, cellulose, alginate, wool, amber, keratin, collagen, starch, DNA, and shellac.

In one embodiment, the drug/therapeutic/active agent can be added to one or more of the substances that cross-link to form the hydrogel. For example, if a hydrogel is formed from two macromers, the drug can be loaded to one of the macromers while in solution, while the other macromer does not contain any drug(s), or contains the same drug/therapeutic/active agent, or another drug/therapeutic/active agent. The drug/therapeutic/active agent(s) may be loaded in the same or varying concentrations in the components/constituents used to form the hydrogel.

In embodiments, the hydrogel forms or dissolves within seconds, minutes, hours, weeks, months, or years, such as up to 1, 10, 20, 30, 50, 60 seconds; up to 1, 2, 3, 4, 5, 6, 7, 8, 9, 10, 15, 20, 25, 30, 40, 45, 50 or minutes; or up to 1, 2, 3, 4, 5, 6, 7, 8, 9, or 10 hours or more; or up to 1, 2, 3, 4, 5, 6, 7, 8, 9, or 10 days or more; or up to 1, 2, 3, 4, 5, 6, 7, 8, 9, or 10 weeks or more; or up to 1, 2, 3, 4, 5, 6, 7, 8, 9, or 10 months or more; or up to 1, 2, 3, 4, 5, 6, 7, 8, 9, or 10 years or more. The rate of polymerization or depolymerization can be tailed for a particular application and depends on various factors such as compositions, component ratios, concentration/weight percentages, solvent composition, drug composition, drug concentration, and other factors as previously described.

In embodiments, the drug is released from the hydrogel over seconds, minutes, hours, weeks, months, or years, such as up to 1, 10, 20, 30, 50, 60 seconds; up to 1, 2, 3, 4, 5, 6, 7, 8, 9, 10, 15, 20, 25, 30, 40, 45, 50 or minutes; or up to 1, 2, 3, 4, 5, 6, 7, 8, 9, or 10 hours or more; or up to 1, 2, 3, 4, 5, 6, 7, 8, 9, or 10 days or more; or up to 1, 2, 3, 4, 5, 6, 7, 8, 9, or 10 weeks or more; or up to 1, 2, 3, 4, 5, 6, 7, 8, 9, or 10 months or more; or up to 1, 2, 3, 4, 5, 6, 7, 8, 9, or 10 years or more, or any range in between using any of these numbers as endpoints for ranges.

In embodiments, the viscosity of the drug-loaded hydrogel ranges from about 0.10 centipoise to about 100,000 centipoise, or any viscosity in between, including 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100,000 centipoise, or any range in between using any of these numbers as endpoints for ranges. In other embodiments, the viscosity of the drug-loaded hydrogel solution ranges from about 1 to about 1,000 centipoise, or from about 1 to 7 Pa*s, such as from about 1 to 3 Pa*s. In other embodiments, the viscosity of the solution ranges from about 1 to about 100 centipoise. It is preferred that the viscosity of the solution is maintained low enough so that it is not too viscous such that the injection cannot be performed with a needle or catheter. The viscosity of the solution can be manipulated by the varying the polymer and/or solvent selected, the polymer concentration, polymer molecular weight, crosslinking, drug choice, drug concentration, or by the addition of additional agents including microbubbles and carbon-based materials, such as graphene.

In one embodiment, the molecular weight of the polymers can be varied from around 1 kDa to 1,000,000 kDa. The molecular weight of the polymer is preferred to be from 10 kDa to 80 kDa. In one example, a high molecular weight can yield small pores in the device and thus, create an effective occlusion. A high molecular weight can also create a more viscous solution and thus, can be more difficult to inject. In other embodiments, the polymers can have a weight average molecular weight (Mw) or number-average molecular weight (Mn) ranging from about 1,000 to 1,000,000 Daltons as measured by GPC (gel permeation chromatography) with polystyrene equivalents, mass spectrometry, or other appropriate methods. The term “about” used herein in the context of quantitative measurements means±10%. For example, with a ±10% range, a number average molecular weight (Mn) or the weight average molecular weight (Mw) of “about 1,000 Daltons” can mean a molecular weight in the range of 900-1,100 Daltons. In embodiments, the number average molecular weight (Mn) or the weight average molecular weight (Mw) of polymers of the invention can range from about 1,000 to about 1,000,000 Daltons, such as from about 3,000 to about 60,000 Daltons, or from about 20,000 to about 90,000 Daltons, or from about 150,000 to about 900,000 Daltons, or from about 200,000 to about 750,000 Daltons, or from about 250,000 to about 400,000 Daltons, or from about 300,000 to about 800,000 Daltons, and so on. Further, the degree of polymerization of the polymers in embodiments can range from 1 to 10,000, such as from 50 to 500, or from 500 to 5,000, or from 1,000 to 3,000.

The molecular weight of substances (e.g., one or more drugs, therapeutics and/or active agents) delivered by the polymer can range from less than 900 Daltons for small molecules to up to 1000 kDa for biologics, for example.

In embodiments, the chain length or degree of polymerization (DP) can have an effect on the properties of the polymers. In the context of this specification, the degree of polymerization is the number of repeating units in the polymer molecule. In embodiments, the polymers include from 2 to about 10,000 repeating units. Preferred are polymers which include from about 5 to 10,000 repeating units, such as from about 10 to 8,000, or from about 15 to 7,000, or from about 20 to 6,000, or from about 25 to 4,000, or from about 30 to 3,000, or from about 50 to 1,000, or from about 75 to 500, or from about 80 to 650, or from about 95 to 1,200, or from about 250 to 2,000, or from about 350 to 2,700, or from about 400 to 2,200, or from about 90 to 300, or from about 100 to 200, or from about 40 to 450, or from about 35 to 750, or from about 60 to 1,500, or from about 70 to 2,500, or from about 110 to 3,500, or from about 150 to 2,700, or from about 2,800 to 5,000, and so on.

If two or more components are used to form the hydrogel or polymeric medical device, the ratio of the components can be varied. The ratio can be 1:1, 2:1, 1:2, 3:1, 1:3, and so on. For example, a 1:1 ratio allows for the highest degree of cross-linking to occur. The ratio determines the rate of crosslinking and thus, gelation of the hydrogel/device.

For occlusion or tissue fillers, the size of the needle or catheter can be chosen based on the estimated size of the body part, organ, duct, cavity/space or lumen from the literature, or determined by imaging the dimensions of the body part, organ, duct, cavity/space or lumen of the subject through ultrasound or other imaging modality. In embodiments, the size of the needle can be between 18 gauge to 34 gauge. In other embodiments, the size of the needle is between 21 gauge and 31 gauge. In other embodiments, the size of the needle is at least 23 gauge, such as between 23 gauge and 29 gauge. In another example, the needle that is used to deliver the injection solution contains bores on the side, which allow for the solution to be excreted around the needle, in addition to the bevel. For sealant or coating applications, the device may be applied using different extrusion approaches, such as through needles, catheters, nozzles, spray applicators, and/or plastic tips. The applicator may be chosen based on factors such as desired application, tissue surface area, coating thickness, and gelation rate.

In one embodiment, the weight percent, or concentration of the components in solution, is varied from around 1% to around 50% of the component in solvent, such as from 1% to 2%, from 2% to 3%, from 3% to 4%, from 4% to 5%, from 5% to 6%, from 6%, to 7%, from 7%, to 8%, from 8% to 9%, from 9% to 10%, and so on. In another embodiment, the weight percent of the macromer is from around 2.5% to around 20% in the solvent, including 6% to around 20%, 7% to around 20%, 8% to around 20%, as so on. The weight percent can affect the mechanical and chemical properties of the polymer, such as increasing or decreasing pore size, viscosity, hardness, elasticity, density, and degradation.

The solvent that the component is dissolved in can be aqueous (water-based) or an organic solvent e.g. DMSO, PEG, ethanol. The final composition may contain excipients for purposes such as increased solubility or quicker dissolution rate. The pH of the composition in solution can be varied from 4 to 9, such as from 4 to 5, 5 to 6, 6 to 7, 7 to 8, and 8 to 9. The pH of the solution can affect the gelation time and stability of the macromer in solution.

In one embodiment, the gelation rate and time of formation of the polymer device varies. Gelation can occur instantaneously, in less than 1 minute, or within 1-10 minutes.

The hydrogel, composition, polymer device or otherwise referred to as the delivered product can be a biomaterial that is formed from multiple biomaterial components and delivered with any delivery system to target locations. A delivered product can be the implant or structure that is at least partially formed with the system by multiple biomaterial components that react together or assemble into higher order structures via covalent and/or non-covalent bonds, and that is delivered by the system. For example, in certain situations, the delivered product can have a storage modulus (delivered G′) and a loss modulus (delivered G″) when the first component and the second component are conveyed out of a delivery member. The ratio of the delivered G″ to the delivered G′ can between about 1/3 and about 3. In some embodiments, the delivered G′ can be greater than the delivered G″ (i.e., a ratio of the delivered G″ to the delivered G′ is less than 1), thus indicating that the delivered product is more solid than liquid. In some embodiments, the components can be formulated such that a viscoelastic substance (and not a liquid substance) is conveyed out of the exit opening of the delivery member. In some embodiments, the hydrogel is conveyed out of the exit opening of the delivery member into a body part, organ, duct, cavity/space or lumen to at least partially or fully occlude the body part, organ, duct, cavity/space or lumen. In some embodiments, the body part, organ, duct, cavity/space or lumen is chosen from an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

The term “biomaterial product,” “delivered biomaterial product,” “delivered product,” “hydrogel” includes any substance that is delivered for contraceptive and/or drug delivery, for example, by any system or delivery device.

In one embodiment, the device/hydrogel/delivered product swells upon contact with one or more fluids inside the body. Swelling allows for the device to secure itself or “lock” within the body part, duct, organ, cavity/space or lumen to form a good occlusion. The device can swell greater than 100%, such as 100-200%, 200-300%, 300-400%, and so on. The greater the device swells, the greater the likelihood of the device allowing fluid to travel through, and for hydrostatic pressure to be reduced. Swelling may also allow for the device to properly secure itself within the body part, duct, organ, cavity/space or lumen.

According to another embodiment, the device includes pores. The pores are homogenous on the surface of the device. The porosity is defined by the properties of the macromers and cross-linking of the macromers. In embodiments, the pore diameter of the formed polymer ranges from 0.001 nm to 3 μm, such as from 0.001 nm to 1 μm. In other embodiments, the pore diameter ranges from 0.01 nm to 100 nm, or from about 1 nm to about 1 μm. In other embodiments, the pore diameter is 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 2, 3, 4, 5, 6, 7, 8, 9, 10, 15, 20, 25, 30, 35, 40, 45, 50, 55, 60, 65, 70, 75, 80, 95, 90, 95, or 100 nm. In other embodiments, the pore diameter is at least the size of an atom (0.5 nm). Specific pore sizes can be targeted to provide an optimum porosity that provides maximum flow of fluid while blocking the flow of sperm cells or ova. In other embodiments, the pores range from 0.1 nm to 2 microns in diameter. In one embodiment, the device is suitable for occlusion of reproductive cells. The pores are less than 3 um to prevent the flow of sperm. The pores allow for fluid to travel through the hydrogel. The mesh size of the device is small enough to block reproductive cells from traversing through. In one embodiment, a larger pore size may be desire for quicker release of drug from the hydrogel.

In embodiments, the length of occlusion produced in a body part, duct, organ, cavity/space or lumen as a result of administering the occlusive substance ranges from 0.1-10 centimeters in length, including 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 cm in length, and so on.

In one embodiment, the hydrogel/device/delivered product does not degrade inside the body in that it is permanent. In another embodiment, the hydrogel/device/delivered product degrades or is capable of degrading in the body, for example, by way of an endogenous stimulus (e.g., hydrolysis). The degradation rate is slow enough that the device remains an effective occlusion inside the body for greater than three months. According to another embodiment, the device degrades upon application of an exogenous stimulus, for example, by photodegradation (e.g., ultraviolet or infrared exposure), acoustic, and/or enzymatic degradation. Alternatively or in addition to any of these embodiments, the hydrogel/device/delivered product is configured to have a lifetime that is as long or longer than the drug/therapeutic agent/active agent release profile.

In one embodiment, a multi-syringe system is used to inject or implant the polymeric device for occlusion. Each syringe can inject a separate macromer/component/constituent. The system can also contain a component that mixes the macromer/component/constituent solutions before implanting into the body and has multiple channels that prevent the components from mixing. The macromer/component/constituent cross-link in situ to form the hydrogel/device, such as an occlusive device. In another aspect, the cross-linking is complete within the injection device prior to the hydrogel being implanted into the body. The injection speed and injection volume can be controlled, tuned, or automated. A handheld device may be used for performing the injection. The injection device can be single use and disposable, or can be multiuse with a replaceable cartridge container in which the macromer solutions are delivered. In one aspect, mixing and/or dissolution of the drug and macromer solution is conducted within the multi-syringe system.

In some embodiments, a delivery system for delivering the hydrogel/device/delivered product can include a container assembly, a connector, a delivery member, and a drive assembly. The container assembly contains a first component and a second component with first component being separate from the second component within the container assembly. The first component is formulated to be crosslinked with the second component to form a hydrogel. The first component and the second component are formulated such that the hydrogel has a gelation time. The connector is configured to be coupled to the container assembly. The delivery member is configured to be coupled to the connector and to be inserted into a body part, organ, duct, cavity/space or lumen. The drive assembly is configured to be operatively coupled to the container assembly. The drive assembly is configured to move a first plunger within the first container to convey at least a portion of the first component from the first container and a second plunger within the second container to convey at least a portion of the second component from the second container. The drive assembly moves the first plunger and the second plunger, independently or together, to convey a portion of the first component and a portion of the second component through the connector and out of the delivery member within a delivery time that is less than the gelation time.

In some embodiments, a method includes coupling a container assembly to a delivery member. The container assembly defines a first chamber and a second chamber, with the first chamber being fluidically isolated from the second chamber. The first chamber contains a first component and the second chamber contains a second component. The first component is formulated to be crosslinked with the second component to form a hydrogel. The first component and the second component are formulated such that the hydrogel has a gelation time. A portion of the first component and a portion of the second component are conveyed into a mixing volume of the delivery member and through the delivery member within a delivery time that is less than the gelation time. The first component crosslinks with the second component to at least partially form the hydrogel/device within the delivery member such that the conveying causes the hydrogel to be conveyed out of an exit opening of the delivery member.

In some embodiments, a composition includes a first component and a second component that are each formulated to be crosslinked with the other to form a hydrogel. The first component and the second component are formulated to have an initial storage modulus (initial G′) and an initial loss modulus (initial G″) when initially combined such that a ratio of the initial G″ to the initial G′ is between about 5 and about 100. The first component and the second component are formulated to have a gelation storage modulus (gelation G′) and a gelation loss modulus (gelation G″) at a gelation time after the first component and the second component are combined such that a ratio of the gelation G″ to the gelation G′ is less than about 5, such as less than about 1. In embodiments, the gelation time is less than about 120 seconds. The term “gelation” refers to the transition of the hydrogel components from a soluble polymer of finite branches to a substance with infinitely large molecules. Similarly stated, “gelation” refers to the condition where the gel forms and after the components are combined. Thus, the gelation time refers to the time that it takes for the resulting hydrogel to substantially reach equilibrium.

In some embodiments, the gelation time is less than about 60 seconds, for example, less than about 30 seconds, and in some cases may be instantaneous/immediate. In other embodiments, the gelation time is between about 1 second and 60 seconds. The particular components used to form the hydrogel/device/delivered product can be selected such that the gelation time/rate is “tuned” for the particular application. For example, the components/constituents can be selected to provide for faster or slower gelation times as desired.

In some embodiments, the ratio of the gelation G″ to the gelation G′ is less than about 0.2, such as about 0.1. In yet other embodiments, the ratio of the gelation G″ to the gelation G′ is a ratio of up to 1, such as a ratio of up to 0.9, or up to 0.8, or up to 0.7, or up to 0.6, or up to 0.5, or up to 0.4, or up to 0.3, or up to 0.2, or up to 0.1.

In some embodiments, a method includes coupling a container assembly to a delivery member. The container assembly defines a first chamber and a second chamber, with the first chamber being fluidically isolated from the second chamber. The first chamber contains a first component and the second chamber contains a second component. The first component is formulated to be crosslinked with the second component to form a hydrogel. The first component and the second component are formulated such that the hydrogel has a gelation time. A portion of the first component and a portion of the second component are conveyed into a mixing volume of the delivery member and through the delivery member within a delivery time that is less than the gelation time. The first component crosslinks with the second component to at least partially form the hydrogel/device within the delivery member such that the conveying causes the hydrogel to be conveyed out of an exit opening of the delivery member.

In some embodiments, the first component and the second component are formulated such that a viscoelastic substance is conveyed out of the exit opening of the delivery member.

In some embodiments, the first component and the second component are formulated to have an initial storage modulus (initial G′) and an initial loss modulus (initial G″) when the first component and the second component are initially combined. A ratio of the initial G″ to the initial G′ is between about 5 and about 100. The first component and the second component are formulated to have a delivered storage modulus (delivered G′) and a delivered loss modulus (delivered G″) when the first component and the second component are conveyed out of the delivery member (e.g., at the delivery time). A ratio of the delivered G″ to the delivered G′ is between about 1/3 and about 3. In some embodiments, the ratio of the initial G″ to the initial G′ is between about 30 and about 5 and the ratio of the delivered G″ to the delivered G′ is between about 1/3 and about 1. In some embodiments, the first component and the second component are formulated to have a gelation storage modulus (gelation G′) and a gelation loss modulus (gelation G″) after the gelation time and a ratio of the gelation G″ to the gelation G′ being less than about 0.2. In some embodiments, the ratio of the gelation G″ to the gelation G′ is about 0.1.

In some embodiments, the first component is at least one of a polyvinyl alcohol, alginate or modified alginate, chitosan or modified chitosan, polyethyleneimine, carboxymethyl cellulose, and/or polyethylene glycol terminated with a biorthogonal functional group (e.g., amine, thiol, maleimide, azide, activated ester). The second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group (e.g., amine, thiol, maleimide, azide, activated ester). In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1 to 30% in solvent. In some embodiments the polysaccharides may be modified with different functional groups. In some embodiments the polysaccharides and proteins may range in molecular weight from 10,000-1,000,000 grams/mole. In some embodiments, the polyvinyl alcohol, polystyrene sulfonate, polyethyleneimine, and polyethylene glycol may be linear, Y-shaped, 3-arm, 4-arm, 6-arm, or 8-arm and range in molecular weight from 1,000-1,000,000 grams/mole.

In some embodiments, the dissolving solution for the polymer component(s) may be aqueous buffers, including any one or more of phosphate, citrate, acetate, histidine, lactate, tromethamine, gluconate, aspartate, glutamate, tartrate, succinate, malic acid, fumaric acid, alpha-ketoglutaric, and/or carbonate. Specific solvents/buffers can include: 1) acetic acid and sodium acetate (AA), 2) citric acid and sodium citrate (CP), 3) citric acid and phosphate buffer (CP), and 4) phosphate buffer (PB), or combinations thereof. Non-aqueous solvents include: dimethyl isosorbide, glycofurol 75, PEG 200, diglyme, tetrahydrofurfuryl alcohol, ethanol, acetone, solketal, glycerol formal, dimethyl sulfoxide, propylene glycol, ethyl lactate, N-methyl-2-pyrrolidone, dimethylacetamide, methanol, isopropanol, 1,4-butanediol, ethyl acetate, toluene, acetonitrile, and combinations thereof.

The molarity of the solutions/solvents/buffers can range for example from 0.1 M to 0.15 M to 0.2 M. In some embodiments, the solution can include a 0.2M citric acid buffer and can be formulated to have a solution pH of between 4.0 and 6.0. In some embodiments, the pH of the solution can be between 4.0 and 5.25, or about 4.0. In other embodiments, the pH of the solution can be about 5.25. In yet other embodiments, the pH of the solution can be between about 4.5 and about 8 such as a pH of about 5-7, or about 4.5-6.

In certain situations, the biomaterial/hydrogel/device/delivered product can be delivered by a delivery system in a fully formed state to a target location. Although a delivered product can be considered fully formed (i.e., the chemical reactions between the biomaterial components are completed), it can still undergo certain changes (e.g., in vivo changes) after delivery. For example, a delivered biomaterial product can continue to absorb water and/or swell and/or can expel impurities. In some embodiments, a delivered biomaterial product can be a hydrogel that is formed by crosslinking of two or more biomaterial components. The term “hydrogel” can refer to any water-swollen (majority, >50%, of material mass is water), and cross-linked polymeric network produced by the reaction of one or more components (e.g., polymers, monomers) and/or a polymeric material that exhibits the ability to swell and retain a significant fraction of water within its structure, but will not dissolve in water.

In embodiments, a first component and a second component can each be a water soluble component (e.g., monomer, macromer, polymer, or the like) that is capable of crosslinking (e.g., with the other component) to form a hydrogel (as the delivered biomaterial product). In some embodiments, the first component and the second component are formulated such that the resulting hydrogel has a gelation time of less than 5 minutes. In other embodiments, the first component and the second component are formulated such that the resulting hydrogel has a gelation time of less than minutes. In other embodiments, the first component and the second component are formulated such that the resulting hydrogel has a gelation time of less than minute. In yet other embodiments, the first component and the second component are formulated such that the resulting hydrogel has a gelation time of less than 30 seconds. In some embodiments, the first component is at least one of a polyvinyl alcohol, alginate or modified alginate, chitosan or modified chitosan, polyethyleneimine, carboxymethyl cellulose, and/or polyethylene glycol terminated with a biorthogonal functional group (e.g., amine, thiol, maleimide, azide, activated ester). The second component is at least one of a water or buffer, water or buffer with divalent cations such as calcium, a solution of reduced hyaluronic acid, a solution of polystyrene sulfonate, a solution of gelatin, and/or polyethylene glycol terminated with a biorthogonal functional group (e.g., amine, thiol, maleimide, azide, activated ester). In some embodiments, polyvinyl alcohol, alginate, chitosan, polyethyleneimine, carboxymethyl cellulose, polyethylene glycol terminated with functional groups, divalent cations, reduced hyaluronic acid, polystyrene sulfonate, or gelatin have a weight percent ranging from about 1 to 30% in solvent. In some embodiments the polysaccharides may be modified with different functional groups. In some embodiments the polysaccharides and proteins may range in molecular weight from 10,000-1,000,000 grams/mole. In some embodiments, the polyvinyl alcohol, polystyrene sulfonate, polyethyleneimine, and polyethylene glycol may be linear, Y-shaped, 3-arm, 4-arm, 6-arm, or 8-arm and range in molecular weight from 1,000-1,000,000 grams/mole. The hydrogel can be any of the hydrogels described herein and can have any of the characteristics as indicated herein. For example, in some embodiments, the formed hydrogel can be at least 90 percent water.

In some embodiments, the biomaterial product/hydrogel can be delivered/introduced to the body/patient at any suitable velocity range. In some embodiments, the predetermined velocity range is bounded by an upper velocity threshold and a lower velocity threshold. By maintaining the velocity below the upper velocity threshold, the biomaterial can be delivered in a manner that limits the likelihood of tissue damage (e.g., due to excessive velocity causing potential tissue damage). Moreover, maintaining the velocity below the upper velocity threshold can ensure that the delivered biomaterial product/hydrogel is formed to the desired extent within the system before exiting the delivery member. For example, in some embodiments, the components that produce the biomaterial product/hydrogel are formulated such that they have an initial storage modulus (initial G′) and an initial loss modulus (initial G″) when the first component and the second component are initially combined. Initially, the G″ is greater than the G′ and the components are in a liquid state when initially combined. For example, certain formulations can produce a ratio of the initial G″ to the initial G′ of between about 5 and about 100. The first component and the second component are further formulated such that after being combined, crosslinking of the components will cause the G′ will increase and will eventually become greater than the G″. In this manner, the components form a viscoelastic (nonliquid) substance. Similarly stated, the crosslinking of the components produces the biomaterial product (e.g., the hydrogel). The time period during which the ratio of G″ to G′ is reduced to a target ratio (e.g., between 1/3 and 3) is dependent on the formulation of the components. Accordingly, by controlling the delivery velocity the system can ensure that the delivered hydrogel is sufficiently formed within the delivery member. For example, in some embodiment, the components are formulated such that the ratio of G″ to G′ reaches a value of less than one in a formation time (e.g., between 6 seconds and 30 seconds). In such embodiments, the desired residence time (i.e., the delivery time) of the first component and the second component within the delivery member is at least as great as the formation time. Additionally, as described here, the components can also have a gelation time that is greater than the delivery time. Thus, the predetermined velocity range can be any suitable range to accommodate the desired delivery characteristics. For example, in some embodiments, the velocity range can be between 0.1 mm/sec to 10 mm/sec. In other embodiments, the velocity range can be between 0.1 mm/sec to 5 mm/sec. In other embodiments, the velocity range can be between 0.01 mm/sec and 1 mm/sec. Moreover, by maintaining the velocity within the predetermined range and for the predetermined delivery time, the system can deliver a volume of the biomaterial product 3 that is within a desired volume range. In this manner, the amount of biomaterial can be accurately controlled. In some embodiments, the volume range is between about 5 microliters and about 1000 microliters. In other embodiments, the volume range is between about 50 microliters and 500 microliters. In yet other embodiments, the volume range is between about 50 microliters and 250 microliters. In still other embodiments, the volume range is between about 75 microliters and 150 microliters.

In some embodiments, the dissolving solution for the polymer component(s) may be aqueous buffers (pH range 1-14), such as phosphate, citrate, acetate, histidine, lactate, tromethamine, gluconate, aspartate, glutamate, tartrate, succinate, malic acid, fumaric acid, alpha-ketoglutaric, and/or carbonate, or combinations thereof. Non-aqueous solvents include: dimethyl isosorbide, glycofurol 75, PEG 200, diglyme, tetrahydrofurfuryl alcohol, ethanol, acetone, solketal, glycerol formal, dimethyl sulfoxide, propylene glycol, ethyl lactate, N-methyl-2-pyrrolidone, dimethylacetamide, methanol, isopropanol, 1,4-butanediol, ethyl acetate, toluene, acetonitrile, and combinations thereof. In some embodiments, when the polymer component is dissolved, the viscosity of the solution(s) that make up the biomaterial may range from 0.1 to 250,000 cP. The density of the solution may range from 0.1 to 20,000 kg/m³. The temperature during extrusion may range from 2 to 45° C. The pH of the solution(s) may range from 1-14. The ionic strength of the solution(s) may range from 1 nM to 70 M.

In some embodiments, if two components are injected to form the biomaterial/hydrogel, then the ratio of the components may be varied such as 1:1, :1, 1:2, 3:1, 1:3, 4:1, 1:4, and up to 10:1 or 1:10. The gelation rate of the biomaterial may range from about 0.001 seconds to 60 minutes. The length of the formed biomaterial may range from about 0.1 to 60 cm. The volume of the formed biomaterial may range from about 0.001 to 100 mL.

In some embodiments, the biomaterial/hydrogel swells within the implantation space to lock or secure its placement. For example, a biomaterial in the form of a hydrogel may swell from about 1.5×-10× its initial volume. In some embodiments, the extruded biomaterial conforms to the space it is injected into. In some embodiments, the swelling of the biomaterial does not change volume within the implantation space, or shrinks to conform to a volume of the implantation space. In some embodiments, the apparatus injects a pre-formed biomaterial (does not cross-link, form, or gel in situ). Once injected, the biomaterial may or may not react with the implantation space. If a reaction does occur, it may be covalent or non-covalent. In some embodiments, the biomaterial adhesively interacts within the implantation space.

In some embodiments, a composition/hydrogel for occulsive effect and/or drug/active agent delivery can include any of the formulations provided in Table 1, and any drug/active agent/therapeutic can be incorporated therein. The formulations provided in Table 1 are merely examples. Based on the information provided therein, one of ordinary skill in the art would be able to interpret the data provided and modify the components of the composition accordingly to achieve a particular objective.

In Table 1, the buffer (first column) for example can include any of Acetic Acid—Sodium Acetate (AA), Citric Acid-Sodium Citrate (CA), Citric Acid (0.2)-Phosphate Buffer (0.1) (CP), or Phosphate Buffer (PB), or combinations thereof. The molarity (M) is provided in the second column, which can be adjusted depending on the embodiment. The pH is provided in the third column, but can be adjusted for any embodiment to have a pH range of about 4-9. The molecular weight (in kDa) is provided in the fourth column, but can also be adjusted such that the polymer has a molecular weight within a desired range. The chemistry of the components is provided in the fifth column, and can include any of the listed combinations including any one or more functional groups chosen from Thiol (SH), Maleimide (MAL), nitrobenzyl (e.g., o-nitrobenzyl, ONB), Hydrazide (HZ), Isocyanate (IC), Amine (NH), Succinimidyl Glutaraldehyde (SG), Aldehyde (AD), or Epoxide (EP), or combinations thereof. The weight percentage (in solution) is provided in the sixth column and likewise can be adjusted according to particular applications, such as providing a composition comprising a desired polymer with a weight percent of up to 20 wt %, such as from about 1-5 wt %, or from about 2-10 wt %, or from about 3-15 wt %, or from about 10-20 wt %. The seventh and eighth columns provide information regarding the testing performed on the formulation, including the method of delivery (seventh column) and the delivery rate (eighth column). Methods of delivery were either via an injection device (VID) similar to those described in U.S. patent application Ser. No. 16/681,572 (published as U.S. Patent Application Publication No. 2020/0146876 and Ser. No. 16/681,577 (U.S. Patent Application Publication No. 2020/0147301), each entitled “Systems and Methods for Delivering of Biomaterials,” and each filed Nov. 12, 2019, each which is incorporated herein by reference in its entirety or via a pipette (PIP). The units of injection rate are microliters per minute (μL/min). The gelation time (seconds) is provided in the last (ninth) column. A gelation time of “Imm.” indicates that gelation occurred immediately after the two components were combined.

TABLE 1
Formulations according to embodiments of the invention

| Buf | M | pH | MW | Chem | Wt % | Meth | Rate | Gel time |
|---|---|---|---|---|---|---|---|---|
| AA | 0.1 | 4.50 | 20 | SH-MAL | 20 | VID | 566 | ~120 |
| AA | 0.1 | 5.00 | 20 | SH-MAL | 20 | VID | 283 | ~120 |
| AA | 0.2 | 4.50 | 20 | SH-MAL | 20 | VID | 5661 | ~120 |
| AA | 0.2 | 4.50 | 20 | SH-MAL | 20 | VID | 566 | ~120 |
| AA | 0.2 | 4.50 | 20 | SH-MAL | 20 | VID | 283 | ~120 |
| AA | 0.2 | 5.00 | 20 | SH-MAL | 20 | VID | 5661 | ~120 |
| AA | 0.2 | 5.00 | 20 | SH-MAL | 20 | VID | 566 | ~120 |
| AA | 0.2 | 5.00 | 20 | SH-MAL | 20 | VID | 283 | ~120 |
| AA | 0.2 | 5.50 | 20 | SH-MAL | 20 | VID | 566 | ~120 |
| AA | 0.2 | 5.50 | 20 | SH-MAL | 20 | VID | 283 | ~120 |
| CA | 0.1 | 4.50 | 20 | SH-MAL | 20 | VID | 5661 | ~120 |
| CA | 0.1 | 4.50 | 20 | SH-MAL | 20 | VID | 566 | ~120 |
| CA | 0.1 | 4.50 | 20 | SH-MAL | 20 | VID | 283 | ~120 |
| CA | 0.1 | 5.00 | 20 | SH-MAL | 20 | VID | 5661 | ~120 |
| CA | 0.1 | 5.00 | 20 | SH-MAL | 20 | VID | 566 | ~120 |
| CA | 0.1 | 5.00 | 20 | SH-MAL | 20 | VID | 283 | ~120 |
| CA | 0.2 | 4.00 | 20 | SH-MAL | 20 | VID | 283 | ~120 |
| CA | 0.2 | 5.25 | 20 | SH-MAL | 20 | VID | 600 | ~30 |
| CA | 0.2 | 5.25 | 20 | SH-MAL | 20 | VID | 566 | ~30 |
| CA | 0.2 | 5.25 | 20 | SH-MAL | 20 | VID | 500 | ~30 |
| CA | 0.2 | 5.25 | 20 | SH-MAL | 20 | VID | 450 | ~30 |
| CA | 0.2 | 5.25 | 20 | SH-MAL | 20 | VID | 400 | ~30 |
| CA | 0.2 | 5.25 | 20 | SH-EMAL | 20 | VID | 400 | ~60 |
| | | | | (ESTER) | | | | |
| CA | 0.2 | 5.25 | 20 | SH-MAL | 20 | VID | 350 | ~30 |
| CA | 0.2 | 5.25 | 20 | SH-MAL | 20 | VID | 300 | ~30 |
| CA | 0.2 | 5.25 | 20 | SH-MAL | 20 | VID | 283 | ~30 |
| CA | 0.2 | 5.25 | 20 | SH-MAL | 15 | VID | 400 | ~30 |
| CA | 0.2 | 5.25 | 20 | SH-MAL | 10 | VID | 400 | ~30 |
| CA | 0.2 | 5.25 | 20 | SH-MAL | 5 | VID | 400 | ~60 |
| CA | 0.2 | 5.25 | 20 | SH-MAL | 2.5 | VID | 400 | ~120 |
| CA | 0.2 | 5.25 | 20 | SH-MAL | 1 | VID | 400 | ~120 |
| CA | 0.2 | 6.00 | 20 | SH-MAL | 2.5 | VID | 400 | ~60 |
| CA | 0.2 | 6.00 | 20 | SH-MAL | 1 | VID | 400 | ~60 |
| CP | 0.15 | 5.50 | 20 | SH-MAL | 20 | VID | 5661 | Imm. |
| CP | 0.15 | 5.50 | 20 | SH-MAL | 20 | VID | 566 | Imm. |
| CP | 0.15 | 5.50 | 20 | SH-MAL | 20 | VID | 283 | Imm. |
| CP | 0.15 | 5.75 | 20 | SH-MAL | 20 | VID | 5661 | Imm. |
| CP | 0.15 | 5.75 | 20 | SH-MAL | 20 | VID | 566 | Imm. |
| CP | 0.15 | 5.75 | 20 | SH-MAL | 20 | VID | 283 | Imm. |
| CP | 0.15 | 5.85 | 20 | SH-MAL | 20 | VID | 5661 | Imm. |
| CP | 0.15 | 5.85 | 20 | SH-MAL | 20 | VID | 566 | Imm. |
| CP | 0.15 | 5.85 | 20 | SH-MAL | 20 | VID | 283 | Imm. |
| PB | 0.1 | 5.50 | 20 | SH-MAL | 20 | VID | 3198 | ~30 |
| PB | 0.1 | 5.50 | 20 | SH-MAL | 20 | VID | 566 | Imm. |
| PB | 0.1 | 5.50 | 20 | SH-MAL | 20 | VID | 283 | Imm. |
| PB | 0.1 | 6.00 | 20 | SH-MAL | 20 | VID | 3198 | Imm. |
| PB | 0.1 | 6.00 | 20 | HZ-AD | 20 | PIP | N/A | Did Not Gel |
| PB | 0.1 | 6.50 | 20 | SH-EP | 20 | PIP | N/A | <24 hrs |
| PB | 0.1 | 6.50 | 20 | SH-MAL | 20 | PIP | N/A | Imm. |
| PB | 0.1 | 6.50 | 40 | SH-MAL | 20 | PIP | N/A | Imm. |
| PB | 0.1 | 6.50 | 20 | SH-MAL | 20 | VID | 5697 | Imm. |
| PB | 0.1 | 6.50 | 20 | SH-MAL | 20 | VID | 5661 | Imm. |
| PB | 0.1 | 6.50 | 20 | SH-MAL | 20 | VID | 3198 | Imm. |
| PB | 0.1 | 6.50 | 20 | SH-MAL | 20 | VID | 2878 | Imm. |
| PB | 0.1 | 6.50 | 20 | SH-MAL | 20 | VID | 2302 | Imm. |
| PB | 0.1 | 6.50 | 20 | SH-MAL | 20 | VID | 1796 | Imm. |
| PB | 0.1 | 6.50 | 20 | SH-MAL | 20 | VID | 1663 | Imm. |
| PB | 0.1 | 6.50 | 20 | SH-MAL | 20 | VID | 1612 | Imm. |
| PB | 0.1 | 6.50 | 20 | SH-MAL | 20 | VID | 1365 | Imm. |
| PB | 0.1 | 6.50 | 20 | SH-MAL | 20 | VID | 1010 | Imm. |
| PB | 0.1 | 6.50 | 20 | SH-MAL | 20 | VID | 566 | Imm. |
| PB | 0.1 | 6.50 | 20 | SH-MAL | 20 | VID | 283 | Imm. |
| PB | 0.1 | 6.50 | 20 | SH-MAL | 15 | PIP | N/A | Imm. |
| PB | 0.1 | 6.50 | 20 | SH-MAL | 10 | PIP | N/A | Imm. |
| PB | 0.1 | 6.50 | 20 | SH-MAL | 5 | PIP | N/A | Imm. |
| PB | 0.1 | 6.50 | 20 | SH-MAL | 2.5 | PIP | N/A | Imm. |
| PB | 0.1 | 6.50(SH) + | 40 | SH-MAL | 15 | PIP | N/A | ~30 |
| | | 7.00 (MAL) | | (ONB) | | | | |
| PB | 0.1 | 7.00 (SG) + | 20 | SG-NH | 20 | PIP | N/A | <24 hrs, |
| | | 9.00 (NH) | | | | | | degraded |
| | | | | | | | | in <3 days |
| | | | | | | | | at 37° C. |
| PB | 0.1 | 7.00 (IC) + | 20 | IC-NH | 20 | PIP | N/A | Did Not Gel |
| | | 9.00 (NH) | | | | | | |
| PB | 0.1 | 7.00 | 20 | SH-MAL | 2.5 | VID | 400 | Imm. |
| PB | 0.1 | 7.00 | 20 | SH-MAL | 1 | VID | 400 | Imm. |
| PB | 0.1 | 8.00 | 20 | SH-MAL | 2.5 | VID | 400 | Imm. |
| PB | 0.1 | 8.00 | 20 | SH-MAL | 1 | VID | 400 | Imm. |
| PB | 0.1 (+0.1/ | 6.50 | 20 | SH-MAL | 20 | PIP | N/A | Imm. |
| | 0.2M NaCl) | | | | | | | |

In one embodiment, a needle or catheter or combination of both can be used to implant the device into the body. For example, if implanting into the vas deferens, a needle must first be used to puncture the thick layers of smooth muscle. However, an angiocath or over-the needle catheter can also be used, which first punctures the vas deferens and then replaces the needle with a catheter. This method can circumvent problems such as the needle puncturing the smooth muscle or extravasating the polymeric material past the body part, duct, organ, cavity/space or lumen. If implanting the device into the fallopian tubes, then a catheter-based approach must typically be used to access the tubes. The gauge of the needle and/or catheter can be chosen based on the dimensions/diameter of the body part, organ, duct, cavity/space or lumen that is being occluded as well as the viscosity of the solutions being injected. Standard needle sizes are readily available such as at: https://www.sigmaaldrich.com/chemistry/stockroom-reagents/learning-center/technical-library/needle-gauge-chart.html.

In one embodiment, the drug-containing hydrogel may be stimulus-responsive, such that upon exposure to one or more stimuli, the hydrogel is reversed and/or drugs are released. The stimulus may cause the hydrogel to dissolve, degrade, de-precipitate, and/or liquefy. In one embodiment, the hydrogel is photoreversible, and the stimulus is light including ultraviolet (UV) or infrared (IR). In one embodiment, the light can be exposed above the skin and penetrate the skin such that the hydrogel is exposed, although infrared (IR) light is able to penetrate skin deeper than ultraviolet (IR). Photodegradation is most effective when the hydrogel is most superficial to the skin. Exposure to light can be accomplished with UV illumination using a UV laser, UV flashlamp, UV fluorescence microscope, or UV fiber optic. A light-emitting diode (LED), violet diode lasers, or a 2-photon light source can be used.

In one embodiment, the ultraviolet light that is used has a defined wavelength. Various wavelengths can impact the release of drugs from the hydrogel. The UV wavelengths can range from 260 nm to 405 nm, or any range in between.

Release of drug from the hydrogel can be accomplished with IR light, including but not limited to, near-infrared, short-wavelength infrared, mid-wavelength infrared, long-wavelength infrared, or far-infrared. The wavelength of the infrared light can range from 700 nanometers to 1100 microns, or any range in between. The frequency of the infrared light can range from 300 GHz to 450 THz, or any range in between.

In one embodiment, the amount of drug released from the hydrogel is dependent upon light intensity. The light intensity can range from 0.1-40 mW/cm². It is preferred that a light intensity of less than 40 J/cm², such as 5-20 mW/cm², is used. Light intensity can be flood-based (non-polarized light) or laser (polarized). Polarized laser light can allow for increased degradation with lower light intensity due to tuning of the wavelength to a specific frequency. Furthermore, lowered light intensity can contribute to a lower degree of potentially adverse cellular effects. The light can be collimated, or can be partially shielded with an opaque photomask to create exposure gradients. The photomask can be moved at various rates e.g. 0.5, 1.2, 2.4 mm/min.

In one embodiment, the amount of drug released from the hydrogel is dependent upon exposure time of the hydrogel to light. Exposure time can range from 1 second to 3,600 seconds. The exposure time is preferably from 1 second to 1,200 seconds. The amount of time can range for example from 10 seconds to 1 minute, up to 2 minutes, or up to 3 minutes, or up to 4 minutes, or up to 5 minutes, or up to 6 minutes, or up to 7 minutes, or up to 8 minutes, or up to 9 minutes, or up to 10 minutes. In one embodiment, the light exposure takes place over the course of one or multiple exposures. The patient can self-activate the release of the drug from the hydrogel. The drug can be released during an in-office visit or during surgery by the physician.

In one embodiment, light is delivered by way of a needle/catheter system such as those described in U.S. Patent Application Publication No. 2019/0038454, which is incorporated by reference herein in its entirety.

In one embodiment, the drug release is expedited via the addition of other external stimuli outside of the exposure of light from the UV or IR spectrum. In one case, this can include addition of physical stimuli (e.g. ultrasound vibration, cavitation, physical manipulation, muscular stimulation, piercing of the occlusion with a needle, catheter, fiber optic, drill, etc.) In one case, this can include the addition of a secondary chemical agent that degrades the hydrogel via secondary chemical means such as enzymatic cleavage, reversal of the crosslinks, ionic solution, pH-altering solution, or addition of some other cleavage factor.

In one embodiment, ultrasound can be used to determine the location of the hydrogel in the body part, organ, duct, cavity/space or lumen, guide the stimulus to the location of the hydrogel, and/or determine if the drug release was successful.

In embodiments, the drug of choice (e.g., active agent, therapeutic) is encapsulated into a drug or cargo carrier. Drug/cargo carriers are species composed of atoms and/or molecules and/or compounds and/or macromolecules that directly contact and/or encapsulate and/or entrap and/or contain the drug or cargo. The drug carriers are then delivered within or by the drug delivery vehicle or depot or hydrogel or gel. The drug delivery vehicle/depot/hydrogel/gel is composed of atoms, molecules, compounds, and/or macromolecules that provide and/or create space within the injection or implantation site for the drug carrier. Drug carriers can be solvents, solutions, polymers, inorganics, proteins, peptides, oligomers, macromers, dendrimers, lipids, amphiphilic compounds, amphiphilic polymers. Examples include but are not limited to the following: solvents/solutions containing the soluble drug/cargo and/or solvents/solutions containing suspensions of the drug/cargo and/or nano- and/or micro-sized materials that are loaded with the drug. Drug loading can be surface bound, encapsulated, evenly distributed, monophasic, biphasic, triphasic, or a combination of these loading mechanisms.

In embodiments, the drug or cargo to be delivered is solubilized in a solvent or solution. That solvent/solution is then mixed with hydrogel precursors or drug delivery depot precursors to create a solution of suspension of pre-hydrogel/drug delivery depot loaded with drug/cargo. Gelation of the hydrogel or formation of the drug delivery depot is induced resulting drug/cargo loaded hydrogel/drug depot. Solvents/solutions include organic solvents such as ethanol, DMSO, propylene glycol, polyethylene glycols, N-methyl-2-pyrrolidone, glycofurol, solketal, glycerol format, acetone, tetrahydrofurfuryl alcohol, diglyme, dimethyl isosorbide, ethyl lactate; aqueous solvents such as water for injection, saline, PBS, histidine buffers, citric acid/citrate buffers, phosphate-citrate buffers, tris buffers, acetate buffers, carbonate buffers, and bicarbonate buffers; or a combination of organic and aqueous solutions listed above.

In embodiments, the drug/cargo carriers may be in the nano-scale range. A nano-scale drug carrier/vehicle is submicron or <1 μm in all dimensions and contains ≥1% drug or cargo by mass. Example compositions of these nanostructures include, but are not limited to, carbon-based structures, carbon allotropes, single-walled nanotubes, fullerenes, buckey balls, inorganics, ceramics, metal, semi-conductive, polymeric, micelles, liposomes, lipid based, nanostars, nanopyramids, nanotubes, nanorods, nanorings, toroids, other nanoarchitectures or combinations of the nanoarchitectures. Nanoarchitectures containing inorganic materials include, but are not limited to gold, metal, platinum, silicon, silicon oxide, calcium oxide, and hydroxyapatitie.

Nano-scale crystals have defined lattice structure of the drug or cargo, in which the drug or cargo compose ≥1% or more of the crystal. Nanoparticulates of drug/cargo are amorphous and are in which the drug or cargo compose ≥1% or more of the crystal as the result of milling, grinding, crushing, precipitation, extruding, or microfluidics.

In embodiments, dendrimers may be used as drug carriers. The physical characteristics of dendrimers, including their monodispersity, water solubility, encapsulation ability, and large number of functionalizable peripheral groups make them attractive drug delivery vehicles.

In other embodiments, micro/micron-scale drug/cargo carriers may be used for the invention. These carriers are greater than or equal to a micron (≥1 μm in one or more dimensions) and contain ≥1% drug or cargo by mass. Examples of micro-architectures and their compositions include, but are not limited to, carbon-based structures, carbon allotropes, inorganics, ceramics, metals, semi-conductive materials, polymers, polymeric materials, fibers, sheets, milled powders, and combinations of the above; micro-architectures containing inorganic materials include, but are not limited to gold, metal, platinum, silicon, silicon oxide, calcium oxide, and hydroxyapatitie; micro-scale crystal (defined lattice structure) of the drug or cargo, in which the drug or cargo compose ≥1% or more of the crystal; and micro-scale particulate (amorphous) of the drug or cargo, in which the drug or cargo compose ≥1% or more of the crystal as the result of milling, grinding, crushing, precipitation, extruding, or microfluidics.

Polymers used in the carrier include, but are not limited to, non-biodegradable polymers such as ester urethanes or epoxy, bis-maleimides, methacrylates such as methyl methacrylate or glycidyl carbonate, tri-methylene carbonate, di-methylene tri-methylene; biodegradable synthetic polymers such as polyglycolic acid, polyglycolide, polylactic acid, 5 polylactide, poly (p-dioxanone), polidioxepanona, poly (oxalates alkylene), modified polyesters such as poly (ether ester) multiblock copolymers such as those based on poly (ethylene glycol) and polybutylene terephthalate; and poly (caprolactone), such as poly (gamma-caprolactone) as well as PCL, PHB, multiblock copolymers of polyether ester, PLGA, PLA, or a combination thereof, for example PLGA, PLA, or a combination of PLA and PLGA. Other species comprises one or more of natural or synthetic monomers, polymers, copolymers or block copolymers, biocompatible monomers, polymers, copolymers or block copolymers, polystyrene, neoprene, polyetherether ketone (PEEK), carbon reinforced PEEK, polyphenylene, polyetherketoneketone (PEKK), polyaryletherketone (PAEK), polyphenylsulphone, polysulphone, polyurethane, polyethylene, low-density polyethylene (L,DPE), linear low-density polyethylene (LLDPE), high-density polyethylene (HDPE), polypropylene, polyetherketoneetherketoneketone (PEKEKK), nylon, fluoropolymers, polytetrafluoroethylene (PTFE or TEFLON®), TEFLON® TFE (tetrafluoroethylene), polyethylene terephthalate (PET or PETE), TEFLON® FEP (fluorinated ethylene propylene), TEFLON® PFA (perfluoroalkoxy alkane), and/or polymethylpentene (PMP) styrene maleic anhydride, styrene maleic acid (SMA), polyurethane, silicone, polymethyl methacrylate, polyacrylonitrile, poly (carbonate-urethane), poly (vinylacetate), nitrocellulose, cellulose acetate, urethane, urethane/carbonate, polylactic acid, polyacrylamide (PAAM), poly (N-isopropylacrylamine) (PNIPAM), poly (vinylmethylether), poly (ethylene oxide), poly (ethyl (hydroxyethyl) cellulose), polyoxazoline and any of its derivatives (POx), polylactide (PLA), polyglycolide (PGA), poly(lactide-co-glycolide) PLGA, poly(e-caprolactone), polydiaoxanone, polyanhydride, trimethylene carbonate, poly(β-hydroxybutyrate), poly(g-ethyl glutamate), poly(DTH-iminocarbonate), poly(bisphenol A iminocarbonate), poly(orthoester) (POE), polycyanoacrylate (PCA), polyphosphazene, polyethyleneoxide (PEO), polyethyleneglycol (PEG) or any of its derivatives, linear or multi-armed PEG and any of its derivatives, polyacrylacid (PAA), polyacrylonitrile (PAN), polyvinylacrylate (PVA), polyvinylpyrrolidone (PVP), polyglycolic lactic acid (PGLA), poly(2-hydroxypropyl methacrylamide) (pHPMAm), poly(vinyl alcohol) (PVOH), PEG diacrylate (PEGDA), poly(hydroxyethyl methacrylate) (pHEMA), N-isopropylacrylamide (NIPA), poly(vinyl alcohol) poly(acrylic acid) (PVOH-PAA), collagen, silk, fibrin, gelatin, hyaluron, cellulose, chitin, dextran, casein, albumin, ovalbumin, heparin sulfate, starch, agar, heparin, alginate, fibronectin, keratin, pectin, elastin, ethylene vinyl acetate, ethylene vinyl alcohol (EVOH), polyethylene oxide, PLA or PLLA (poly(L-lactide) or poly(L-lactic acid)), poly(D,L-lactic acid), poly(D,L-lactide), polydimethylsiloxane or dimethicone (PDMS), poly(isopropyl acrylate) (PIPA), polyethylene vinyl acetate (PEVA), PEG styrene, polytetraflurorethylene RFE, TEFLON® RFE, KRYTOX® RFE, fluorinated polyethylene (FLPE or NALGENE®, methyl palmitate, temperature responsive polymers, poly(N-isopropylacryl amide) (NIPA), polycarbonate, polyethersulfone, polycaprolactone, polymethyl methacrylate, polyisobutylene, nitrocellulose, medical grade silicone, cellulose acetate, cellulose acetate butyrate, polyacrylonitrile, poly(lacti de-co-caprolactone (PLCL), and/or chitosan, or combinations thereof.

Synthetic non-degradable polymers used in the drug-carrier include, but are not limited to, polyethylene, acetate, polyethylene-co-vinyl), polypropylene, polyvinyl chloride, polyvinyl acetate, polyvinyl alcohol and copolymers of vinyl alcohol and vinyl acetate, polyacrylic acid, polymethacrylic acid), polyacrylamides, polymethacrylamides, polyacrylates, poly (ethylene glycol), poly (dimethylsiloxane), polyurethanes, polycarbonates, polystyrene and derivatives and combinations thereof.

Natural polymers used in the drug-carrier/vehicle include, but are not limited to, carbohydrates, polypeptides and proteins including starch, cellulose and derivatives including ethylcellulose, methylcellulose, ethylhydroxy ethylcellulose, sodium carboxymethylcellulose; collagen; jelly; dextran and derivatives; alginates; chitin; and chitosan and combinations thereof.

Lipid based vehicles or liposomes may comprise, but are not limited to: 1,2-distearoyl-sn-glycero-3-phosphocholine (DSPC), 1,2-dioleoyl-sn-glycero-3-phosphocholine (DOPC), cholesterol, and 1,2-dipalmitoyl-sn-glycero-3-phospho-(1′-rac-glycerol) (DPPG) as well as derivatives. Suitable fatty acid (e.g. esters) include those that can be derived from fatty acids and hydroxyl fatty acids defined above. Preferred esters are fatty acid monoesters and diesters of fatty acids and derivatives thereof such as mono- and diesters of polyethylene glycol (PEG) fatty acid. Suitable PEG include those having from 2 to 200 monomer units, preferably 4 to 100 monomer units, for example 10 to 15 monomer units. Examples include PEG stearate and PEG distearate, such as PEG-40 stearate (Crodet S40, Croda) and PEG-8 distearate (Lipopeg 4-DS, Adina). Additional examples of fatty acid derivatives include fatty acids esterified with polyoxyethylene sorbitan compounds, such as “Tween” (monooleate example polyoxyethylene (20) sorbitan, also known as Tween 80) and fatty acids esterified with sorbitan compounds, such as “Span” (for example sorbitan monooleate, also known as Span 80), or combinations thereof.

The micron-sized carrier may also include combinations of the above listed species. These combinations include monophasic, biphasic, and triphasic compositions. These types of combinations include but are not limited to, metal (gold, silver, platinum)-core-silicone coated particles, solid lipid particles. The drug/cargo carrier can also be a combination of the above listed nano- and micro-species.

The drug delivery vehicle/depot/hydrogel/gel are species composed of atoms, molecules, compounds, and/or macromolecules that provide and/or create space within the injection or implantation site for the drug carrier(s). Vehicles can create space for drug carrier, and enable them to diffuse or degrade. Additionally, vehicles can form a network (greater than the random association of solvent molecules) within the injection of implantation space that entraps, encapsulates, contains, prevents diffusion, and/or prevents aggregation of the drug carrier.

Vehicles can include, but are not limited to gases, solutions/solvents, lipids, amphiphilics, polymers, hydrogels, and suspensions. Gases include but are not limited to nitrogen, argon, sterile air. Solvents/solutions include but are not limited to organic solvents (i.e. ethanol, DMSO, propylene glycol, polyethylene glycols, N-methyl-2-pyrrolidone, glycofurol, solketal, glycerol format, acetone, tetrahydrofurfuryl alcohol, diglyme, dimethyl isosorbide, ethyl lactate), aqueous solutions (i.e. water for injection, saline, PBS, histidine buffers, citric acid/citrate buffers, phosphate-citrate buffers, tris buffers, acetate buffers, carbonate buffers, bicarbonate buffers), organic/aqueous solutions, or combinations of any of these solvents/solutions.

In embodiments, the drug carrier may be cross-linked to the polymer and/or hydrogel.

In embodiments, the invention may be used for prevention (prophylaxis) and/or treatment of STIs (for example, using any methods and/or compositions of the invention, including any of Aspects 1-92 above or any of Examples 1-14 below), including, but not limited to, prevention and/or treatment of bacterial vaginosis (BV), chlamydia, gonorrhea, hepatitis, such as hepatitis B (HBV) or hepatitis C, herpes, such as HSV1 or HSV2, ebola virus, human immunodeficiency virus or acquired immune deficiency syndrome (HIV/AIDS), human papilloma virus (HPV), pelvic inflammatory disease (PID), nongonoccocal urethritis (NGU), syphilis, trichomoniasis, disease or infection caused by Haemophilus ducreyi, such as chancroid, methicillin-resistant Staphylococcus aureus (MRSA), lymphogranuloma venereum (LGV), Mycoplasma genitalium (MG), crabs or pubic lice or infestation of such, molluscum contagiosum, and scabies, or combinations thereof.

In embodiments, the invention may be used for the prevention and/or treatment of other bacterially-caused diseases affecting genitourinary organs (for example, using any methods and/or compositions of the invention, including any of Aspects 1-92 above or any of Examples 1-14 below), including preventing/treating urinary tract infections, mycoplasmal and ureaplasmal urethritis, and Lymphogranuloma venereum, or combinations thereof.

The invention may be used for occlusion (for example, using any methods and/or compositions of the invention, including any of Aspects 1-92 above or any of Examples 1-14 below) of the femoral artery, popliteal artery, coronary and/or carotid artery; the esophagus, the oral cavity, nasopharyngeal cavity, ear canal and tympanic cavity, sinuses of the brain, the arterial system, the venous system, heart, larynx, trachea, bronchi, stomach, duodenum, ileum, colon, rectum, bladder, kidney, ureter, ejaculatory duct, epididymis, vas deferens, the urethra, the uterine cavity, a vaginal canal, fallopian tubes, and cervix; any duct including a bile duct, a hepatic duct, a cystic duct, a pancreatic duct, or a parotid duct; an organ including a uterus, prostate, or any organ of the gastrointestinal tract or circulatory system or respiratory system or nervous system, or urological organ, or combinations thereof.

The invention may be used as a drug depot (for example, using any methods and/or compositions of the invention, including any of Aspects 1-92 above or any of Examples 1-14 below) in the subcutaneous space, interstitial space, intramuscular, rectum, and peritoneal cavity, or combinations thereof.

The invention can also be injected as a bulking agent for hard tissue defects (for example, using any methods and/or compositions of the invention, including any of Aspects 1-92 above or any of Examples 1-14 below), such as bone or cartilage defects, or combinations thereof.

The invention may be used for treatment of one or more cancer (for example, using any methods and/or compositions of the invention, including any of Aspects 1-92 above or any of Examples 1-14 below) to treat breast cancer, colon cancer, rectal cancer, endometrial cancer, cervical cancer, kidney cancer, leukemia, liver cancer, stomach cancer, esophageal cancer, oral cancer, throat cancer, tracheal cancer, lung cancer, melanoma, non-melanoma skin cancers, non-Hodgkin lymphoma, Hodgkin lymphoma, pancreatic cancer, prostate cancer, head and neck cancers, bone cancer, and thyroid cancer, or combinations thereof.

The invention (for example, using any methods and/or compositions of the invention, including any of Aspects 1-92 above or any of Examples 1-14 below) may be used for treatment of central nervous system (CNS)-related diseases and conditions (which encompass psychiatric/behavioral diseases or disorders), including, without limitation, acquired epileptiform aphasia, acute disseminated encephalomyelitis, adrenoleukodystrophy, agenesis of the corpus callosum, agnosia, aicardi syndrome, Alexander disease, Alpers' disease, alternating hemiplegia, Alzheimer's disease, amyotrophic lateral sclerosis, anencephaly, Angelman syndrome, angiomatosis, anoxia, aphasia, apraxia, arachnoid cysts, arachnoiditis, Arnold-chiari malformation, arteriovenous malformation, Asperger's syndrome, ataxia telangiectasia, attention deficit hyperactivity disorder, autism, auditory processing disorder, autonomic dysfunction, back pain, Batten disease, Behcet's disease, Bell's palsy, benign essential blepharospasm, benign focal amyotrophy, benign intracranial hypertension, bilateral frontoparietal polymicrogyria, binswanger's disease, blepharospasm, Bloch-sulzberger syndrome, brachial plexus injury, brain abscess, brain damage, brain injury, brain tumor, spinal tumor, Brown-sequard syndrome, canavan disease, carpal tunnel syndrome (cts), causalgia, central pain syndrome, central pontine myelinolysis, centronuclear myopathy, cephalic disorder, cerebral aneurysm, cerebral arteriosclerosis, cerebral atrophy, cerebral gigantism, cerebral palsy, charcot-marie-tooth disease, chiari malformation, chorea, chronic inflammatory demyelinating polyneuropathy (“CIDP”), chronic pain, chronic regional pain syndrome, Coffin lowry syndrome, coma (including persistent vegetative state), congenital facial diplegia, corticobasal degeneration, cranial arteritis, craniosynostosis, Creutzfeldt-jakob disease, cumulative trauma disorders, Cushing's syndrome, cytomegalic inclusion body disease (“CIBD”), cytomegalovirus infection, dandy-walker syndrome, Dawson disease, de morsier's syndrome, Dejerine-klumpke palsy, Dejerine-sottas disease, delayed sleep phase syndrome, dementia, dermatomyositis, developmental dyspraxia, diabetic neuropathy, diffuse sclerosis, dysautonomia, dyscalculia, dysgraphia, dyslexia, dystonia, early infantile epileptic encephalopathy, empty sella syndrome, encephalitis, encephalocele, encephalotrigeminal angiomatosis, encopresis, epilepsy, Erb's palsy, erythromelalgia, essential tremor, Fabry's disease, Fahr's syndrome, fainting, familial spastic paralysis, febrile seizures, fisher syndrome, Friedreich's ataxia, Gaucher's disease, Gerstmann's syndrome, giant cell arteritis, giant cell inclusion disease, globoid cell leukodystrophy, gray matter heterotopia, Guillain-barré syndrome, htiv-1 associated myelopathy, Hallervorden-spatz disease, head injury, headache, hemifacial spasm, hereditary spastic paraplegia, heredopathia atactica polyneuritiformis, herpes zoster oticus, herpes zoster, hirayama syndrome, holoprosencephaly, Huntington's disease, hydranencephaly, hydrocephalus, hypercortisolism, hypoxia, immune-mediated encephalomyelitis, inclusion body myositis, incontinentia pigmenti, infantile phytanic acid storage disease, infantile refsum disease, infantile spasms, inflammatory myopathy, intracranial cyst, intracranial hypertension, Joubert syndrome, Kearns-sayre syndrome, Kennedy disease, kinsbourne syndrome, Klippel feil syndrome, Krabbe disease, Kugelberg-welander disease, kuru, lafora disease, Lambert-eaton myasthenic syndrome, Landau-kleffner syndrome, lateral medullary (Wallenberg) syndrome, learning disabilities, leigh's disease, Lennox-gastaut syndrome, Lesch-nyhan syndrome, leukodystrophy, lewy body dementia, lissencephaly, locked-in syndrome, Lou Gehrig's disease, lumbar disc disease, lyme disease-neurological sequelae, machado-joseph disease (spinocerebellar ataxia type 3), macrencephaly, megalencephaly, Melkersson-rosenthal syndrome, Ménière's disease, meningitis, Menkes disease, metachromatic leukodystrophy, microcephaly, migraine, Miller Fisher syndrome, mini-strokes, mitochondrial myopathies, mobius syndrome, monomelic amyotrophy, motor neurone disease, motor skills disorder, moyamoya disease, mucopolysaccharidoses, multi-infarct dementia, multifocal motor neuropathy, multiple sclerosis, multiple system atrophy with postural hypotension, muscular dystrophy, myalgic encephalomyelitis, myasthenia gravis, myelinoclastic diffuse sclerosis, myoclonic encephalopathy of infants, myoclonus, myopathy, myotubular myopathy, myotonia congenita, narcolepsy, neurofibromatosis, neuroleptic malignant syndrome, neurological manifestations of aids, neurological sequelae of lupus, neuromyotonia, neuronal ceroid lipofuscinosis, neuronal migration disorders, niemann-pick disease, non 24-hour sleep-wake syndrome, nonverbal learning disorder, O'sullivan-mcleod syndrome, occipital neuralgia, occult spinal dysraphism sequence, ohtahara syndrome, olivopontocerebellar atrophy, opsoclonus myoclonus syndrome, optic neuritis, orthostatic hypotension, overuse syndrome, palinopsia, paresthesia, Parkinson's disease, paramyotonia congenita, paraneoplastic diseases, paroxysmal attacks, parry-romberg syndrome (also known as rombergs syndrome), pelizaeus-merzbacher disease, periodic paralyses, peripheral neuropathy, persistent vegetative state, pervasive developmental disorders, photic sneeze reflex, phytanic acid storage disease, pick's disease, pinched nerve, pituitary tumors, polymicrogyria (PMG), polio, polymicrogyria, polymyositis, porencephaly, post-polio syndrome, postherpetic neuralgia (“PHN”), postinfectious encephalomyelitis, postural hypotension, Prader-willi syndrome, primary lateral sclerosis, prion diseases, progressive hemifacial atrophy (also known as Romberg's syndrome), progressive multifocal leukoencephalopathy, progressive sclerosing poliodystrophy, progressive supranuclear palsy, pseudotumor cerebri, ramsay-hunt syndrome (type I and type II), Rasmussen's encephalitis, reflex sympathetic dystrophy syndrome, refsum disease, repetitive motion disorders, repetitive stress injury, restless legs syndrome, retrovirus-associated myelopathy, rett syndrome, Reye's syndrome, Romberg's syndrome, rabies, Saint Vitus' dance, Sandhoff disease, schizophrenia, Schilder's disease, schizencephaly, sensory integration dysfunction, septo-optic dysplasia, shaken baby syndrome, shingles, Shy-drager syndrome, Sjögren's syndrome, sleep apnea, sleeping sickness, snatiation, Sotos syndrome, spasticity, spina bifida, spinal cord injury, spinal cord tumors, spinal muscular atrophy, spinal stenosis, Steele-richardson-olszewski syndrome, see progressive supranuclear palsy, spinocerebellar ataxia, stiff-person syndrome, stroke, Sturge-weber syndrome, subacute sclerosing panencephalitis, subcortical arteriosclerotic encephalopathy, superficial siderosis, sydenham's chorea, syncope, synesthesia, syringomyelia, tardive dyskinesia, Tay-sachs disease, temporal arteritis, tetanus, tethered spinal cord syndrome, Thomsen disease, thoracic outlet syndrome, tic douloureux, Todd's paralysis, Tourette syndrome, transient ischemic attack, transmissible spongiform encephalopathies, transverse myelitis, traumatic brain injury, tremor, trigeminal neuralgia, tropical spastic paraparesis, trypanosomiasis, tuberous sclerosis, vasculitis including temporal arteritis, Von Hippel-lindau disease (“VHL”), Viliuisk encephalomyelitis (“VE”), Wallenberg's syndrome, Werdnig-hoffman disease, west syndrome, whiplash, Williams syndrome, Wilson's disease, and Zellweger syndrome, or combinations thereof.

The invention may be used for treatment of cardiovascular diseases (for example, using any methods and/or compositions of the invention, including any of Aspects 1-92 above or any of Examples 1-14 below), such as hypertension and cardiac insufficiency, stable and unstable angina pectoris and peripheral and cardiac vascular diseases and arrhythmias, hyperlipidemia, hypercholesterolemia, thromboembolic diseases and ischaemias, such as myocardial infarction, cerebral stroke, transitory and ischaemic attacks and peripheral circulatory disturbances, for preventing restenoses, such as after thrombolysis treatment, percutaneous transluminal angioplasties (PTA), percutaneous transluminal coronary angioplasties (PTCA) and bypass, arteriosclerosis and diseases of the urogenital system, such as, for example, prostate hypertrophy, erectile dysfunction and incontinence, or combinations thereof.

The invention (for example, using any methods and/or compositions of the invention, including any of Aspects 1-92 above or any of Examples 1-14 below) may be used for treatment of metabolic syndrome, type I or type II diabetes, pre-diabetes, or obesity, or combinations thereof.

The invention (for example, using any methods and/or compositions of the invention, including any of Aspects 1-92 above or any of Examples 1-14 below) may be used for treatment of infections such as bacterial infections, viral infections, fungal infections, protozoan infections, yeast infections, or parasitic infections, or combinations thereof.

The invention (for example, using any methods and/or compositions of the invention, including any of Aspects 1-92 above or any of Examples 1-14 below) may be used for treatment of any disease of the eye, ear, nose, throat, mouth, lung, heart, liver kidney, spleen, pancreas, gastrointestinal system, circulatory system, reproductive system, central nervous system, immune system, musculoskeletal system, and skin, or combinations thereof.

The drug(s) contained within the hydrogel or drug carrier (for example, according to any methods and/or compositions of the invention, including any of Aspects 1-92 above or any of Examples 1-14 below) may include one or more antibiotics including, but not limited to, amoxicillin, doxycycline, cephalexin, ciprofloxacin, clindamycin, metronidazole, azithromycin, sulfamethoxazole, trimethoprim, amoxicillin, clavulanate, levofloxacin, ampicillin, piperacillin, amoxicillin/clavulanate, ampicillin/sulbactam, piperacillin/tazobactam, penicillin g benzathine, Penicillin V, dicloxacillin, nafcillin, oxacillin, demeclocycline, tetracycline, minocycline, eravacycline, omadacycline, sarecycline, cefadroxil, cefazolin, cephadrine, cephalexin, cefotetan, cefoxitin, cefprozil, cefuroxime, loracarbef, cefdinir, cefditoren, cefixime, cefoperazone, cefotaxime, cefpodoxime, ceftazidime, ceftibuten, ceftriaxone, cefepime, cefpirome, and ceftaroline, cinoxacin, ciprofloxacin, delafloxacin, gemifloxacin, levofloxacin, moxifloxacin, norfloxacin, ofloxacin, sparfloxacin, lincomycin. clindamycin, telithromycin, azithromycin, fidaxomicin, erythromycin, clarithromycin, sulfamethoxazole/trimethoprim, sulfadiazine, sulfisoxazole, dalbavancin, oritavancin, teicoplanin, telavancin, vancomycin, tobramycin, paromomycin, gentamicin, amikacin, neomycin, amikacin, kanamycin, plazomicin, streptomycin, doripenem, meropenem, ertapenem, and cilastatin/imipenem and combinations thereof and/or in combination with any other active agent(s).

The drug(s) contained within the hydrogel or drug carrier (for example, according to any methods and/or compositions of the invention, including any of Aspects 1-92 above or any of Examples 1-14 below) may include one or more antimicrobial agents including, but not limited to, acyclovir, aminoglycosides, gentamicin, tobramycin, amoxicillin, A amoxicillin+clavulanate, amphotericin B, ampicillin, ampicillin/penam sulfones, atovaquone, azithromycin, cefazolin, cephalosporins flat, cefotaxime, cefotetan, cefpodoxime, ceftazidime, cefazolin oxime, ceftriaxone, cefuroxime, cephalexin, chloramphenicol, clotrimazole, ciprofloxacin, clarithromycin, clindamycin, dapsone, dicloxacillin, doxycycline, erythromycin, fluconazole, foscarnet, ganciclovir, gatifloxacin, imipenem, cilastatin, imipenem+cilastatin, isonicotinate corpus, itraconazole miconazole, ketoconazole, metronidazole, nafcillin, nafcillin, nystatin, penicillin, penicillin G, pentane pulse, piperacillin, tazobactam, tazobactam+piperacillin, rifampicin, quinupristin, dalfopristin, quinupristin+dalfopristin, ticarcillin, clavulanate, clarithromycin ticarcillin Potassium, trimethoprim, sulfamethoxazole isoxazole, trimethoprim+sulfamethoxazole isoxazole, Wan valacyclovir, vancomycin, Myron methanesulfonamide, silver sulfadiazine, mupirocin, nystatin suspension, triamcinolone, nystatin, triamcinolone+nystatin, clotrimazole+betamethasone, butoconazole, miconazole, tioconazole, destroying microbes or microbial loss ability detergent substances, nonoxynol-9, octoxynol-9, benzalkonium chloride, n-docosanol and combinations thereof and/or in combination with any other active agent(s).

The hydrogel or drug carrier (for example, according to any methods and/or compositions of the invention, including any of Aspects 1-92 above or any of Examples 1-14 below) may include one or more drugs against herpes including, but not limited to, acyclovir (Zovirax), famciclovir (Famvir), and valacyclovir (Valtrex). The hydrogel or drug carrier may include one or more drugs against syphilis including, but not limited to, penicillin, doxycycline, azithromycin, and ceftriaxone. Combination drugs, which include abacavir/dolutegravir/lamivudine (Triumeq), dolutegravir/rilpivirine (Juluca), elvitegravir/cobici stat/emtricitabine/tenofovir di soproxil fumarate (Stribild), elvitegravir/cobici stat/emtricitabine/tenofovir alafenamide (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy); integrase inhibitors, which include dolutegravir (Tivicay), elvitegravir (Vitekta), raltegravir (Isentress), raltegravir extended-release (Isentress HD); nucleoside/nucleotide reverse transcriptase inhibitors (NRTIs), which include abacavir (Ziagen), abacavir/lamivudine (Epzicom), abacavir/lamivudine/zidovudine (Trizivir), lamivudine/zidovudine (Combivir), lamivudine (Epivir), zidovudine (Retrovir), emtricitabine/tenofovir disoproxil fumarate (Truvada), emtricitabine (Emtriva), tenofovir di soproxil fumarate (Vi read), emtricitabine/tenofovir alafenamide (Descovy); non-nucleoside reverse transcriptase inhibitors (NNRTIs), which include efavirenz (Sustiva), etravirine (Intelence), nevirapine (Viramune), nevirapine extended-release (Viramune XR), rilpivirine (Edurant), delavirdine mesylate (Rescriptor); protease inhibitors, which include atazanavir/cobici stat (Evotaz), darunavir/cobici stat (Prezcobix), lopinavir/ritonavir (Kaletra), ritonavir (Norvir), atazanavir (Reyataz), darunavir (Prezista), fosamprenavir (Lexiva), tipranavir (Aptivus), nelfinavir (Viracept), indinavir (Crixivan), saquinavir (Invirase); entry inhibitors, such as enfuvirtide (Fuzeon); and CCRS antagonists, such as maraviroc (Selzentry) and combinations thereof and/or in combination with any other active agent(s).

The hydrogel or drug carrier (for example, according to any methods and/or compositions of the invention, including any of Aspects 1-92 above or any of Examples 1-14 below) may include one or more non-HIV antivirals such as peramivir, zanamivir, oseltamivir phosphate, acyclovir, famciclovir, and valacyclovir, entecavir, tenofovir, lamivudine, adefovir and telbivudine and combinations thereof and/or in combination with any other active agent(s).

The hydrogel or drug carrier (for example, according to any methods and/or compositions of the invention, including any of Aspects 1-92 above or any of Examples 1-14 below) may include one or more vaccines against HPV, hepatitis A and hepatitis B and combinations thereof and/or in combination with any other active agent(s).

The hydrogel or drug carrier (for example, according to any methods and/or compositions of the invention, including any of Aspects 1-92 above or any of Examples 1-14 below) may include one or more Hepatitis C drugs such as Daclatasvir, Elbasvir-Grazoprevir, Glecaprevir-Pibrentasvir, Ledipasvir-Sofosbuvir, Peginterferon alfa-2a, Peginterferon alfa-2b, Ribavirin, Simeprevir, and Sofosbuvir and combinations thereof and/or in combination with any other active agent(s).

In embodiments, the invention may deliver steroids (for example, using any methods and/or compositions of the invention, including any of Aspects 1-92 above or any of Examples 1-14 below) such as for testosterone replacement therapy to return serum testosterone levels to within physiologic range and improve symptoms in hypogonadal men. Some of the symptoms aimed to improve upon include decreased libido, erectile dysfunction, infertility, hot flashes, depressed mood, and loss of muscle mass or hair. The steroids may also be used to stop the production of sperm in the testes (for male contraception). Steroids include testosterone, testosterone undecanoate, testosterone cypionate, testosterone enanthate, 17α-methyltestosterone, progestins, nesterone, dehydroepiandrosterone (DHEA), dehydroepiandrosterone sulfate (DHEA-S), androstenedione (A4), Corticosteroids and analogues, androstenediol (A5), androsterone, dihydrotestosterone, estrone (E1), estradiol (E2), estriol (E3) and combinations thereof and/or in combination with any other active agent(s).

In embodiments, the invention (for example, using any methods and/or compositions of the invention, including any of Aspects 1-92 above or any of Examples 1-14 below) may deliver the molecules lupeol, pristimerin, niclosamide, GnRH agonists, and/or sirolimus (Rapamycin) and combinations thereof and/or in combination with any other active agent(s).

In embodiments, the invention (for example, using any methods and/or compositions of the invention, including any of Aspects 1-92 above or any of Examples 1-14 below) may deliver small molecules or biologics that affect sperm motility, sperm viability, sperm activation, or prevent fertilization with oocytes. These molecules may have an effect on sperm and/or egg targets including, but not limited to HIPK4, TS SK1-6, SAS1B, SLLP1, JUNO/IZUMO, CatSper, TRPV4, ABHD2, EPPIN, Defensin B126, Slo1/Slo3/LRRC52, Na/K-ATPase alpha 4, sNHE, Hv1, and PPP3CC and PPP3R2 and combinations thereof and/or in combination with other active agent(s).

In embodiments, the invention (for example, using any methods and/or compositions of the invention, including any of Aspects 1-92 above or any of Examples 1-14 below) may deliver spermicidal agents, fertility agents, hormones, growth factors, anti-inflammatory drugs, anti-bacterial agents, anti-viral agents, adherent proteins, contrast agents, imaging agents, therapeutic drugs, antimicrobials, anti-inflammatories, spermicidal agents, vasodilators, steroids, ionic solutions, proteins, nucleic acids, antibodies, or fragments thereof and combinations thereof and/or in combination with any other active agent(s).

In embodiments, the invention may deliver cancer chemotherapeutic agents (for example, using any methods and/or compositions of the invention, including any of Aspects 1-92 above or any of Examples 1-14 below), including Abiraterone Acetate, ABITREXATE (Methotrexate), ABRAXANE (Paclitaxel Albumin-stabilized Nanoparticle Formulation), ADCETRIS (Brentuximab Vedotin), Ado-Trastuzumab Emtansine, ADRIAMYCIN (Doxorubicin Hydrochloride), ADRUCIL (Fluorouracil), Afatinib Dimaleate, AFINITOR (Everolimus), ALDARA (Imiquimod), Aldesleukin, Alemtuzumab, ALIMTA (Pemetrexed Disodium), ALOXI (Palonosetron Hydrochloride), AMBOCHLORIN (Chlorambucil), AMBOCLORIN (Chlorambucil), Aminolevulinic Acid, Anastrozole, Aprepitant, AREDIA (Pamidronate Disodium), ARIMIDEX (Anastrozole), AROMASIN (Exemestane), ARRANON (Nelarabine), Arsenic Trioxide, ARZERRA (Ofatumumab), Asparaginase Erwinia chrysanthemi, AVASTIN (Bevacizumab), Axitinib, Azacitidine, Bendamustine Hydrochloride, Bevacizumab, Bexarotene, BEXXAR (Tositumomab and I 131 Iodine Tositumomab), Bleomycin, Bortezomib, BOSULIF (Bosutinib), Cabazitaxel, Cabozantinib-S-Malate, CAM PATH (Alemtuzumab), CAMPTOSAR (Irinotecan Hydrochloride), Capecitabine, Carboplatin, Carfilzomib, CEENU (Lomustine), CERUBIDINE (Daunorubicin Hydrochloride), Cetuximab, Chlorambucil, Cisplatin, CLAFEN (Cyclophosphamide), Clofarabine, COMETRIQ (Cabozantinib-S-Malate), COSMEGEN (Dactinomycin), Crizotinib, Cyclophosphamide, CYFOS (Ifosfamide), Cytarabine, Dabrafenib, Dacarbazine, DACOGEN (Decitabine), Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, EFUDEX (Fluorouracil), ELITEK (Rasburicase), ELLENCE (Epirubicin Hydrochloride), ELOXATIN (Oxaliplatin), Eltrombopag Olamine, EMEND (Aprepitant), Enzalutamide, Epirubicin Hydrochloride, ERBITUX (Cetuximab), Eribulin Mesylate, ERIVEDGE (Vismodegib), Erlotinib Hydrochloride, ERWINAZE (Asparaginase Erwinia chrysanthemi), Etoposide, Everolimus, EVISTA (Raloxifene Hydrochloride), Exemestane, FARESTON (Toremifene), FASLODEX (Fulvestrant), FEMARA (Letrozole), Filgrastim, FLUDARA (Fludarabine Phosphate), Fludarabine Phosphate, FLUOROPLEX (Fluorouracil), Fluorouracil, Folinic acid, FOLOTYN (Pralatrexate), Fulvestrant, Gefitinib, Gemcitabine Hydrochloride, Gemtuzumab Ozogamicin, GEMZAR (Gemcitabine Hydrochloride), GILOTRIF (Afatinib Dimaleate), GLEEVEC (Imatinib Mesylate), HALAVEN (Eribulin Mesylate), HERCEPTIN (Trastuzumab), HYCAMTIN (Topotecan Hydrochloride), Ibritumomab Tiuxetan, ICLUSIG (Ponatinib Hydrochloride), Ifosfamide, Imatinib Mesylate, Imiquimod, INLYTA (Axitinib), INTRON A (Recombinant Interferon Alfa-2b), Iodine 131 Tositumomab and Tositumomab, Ipilimumab, IRES SA (Gefitinib), Irinotecan Hydrochloride, ISTODAX (Romidepsin), Ixabepilone, JAKAFI (Ruxolitinib Phosphate), JEVTANA (Cab azitaxel), Kadcyla (Ado-Trastuzumab Emtansine), KEOXIFENE (Raloxifene Hydrochloride), KEPIVANCE (Palifermin), KYPROLIS (Carfilzomib), Lapatinib Ditosylate, Lenalidomide, Letrozole, Leucovorin Calcium, Leuprolide Acetate, Lomustine, LUPRON (Leuprolide Acetate, MARQIBO (Vincristine Sulfate Liposome), MATULANE (Procarbazine Hydrochloride), Mechlorethamine Hydrochloride, MEGACE (Megestrol Acetate), Megestrol Acetate, MEKINIST (Trametinib), Mercaptopurine, Mesna, METHAZOLASTONE (Temozolomide), Methotrexate, Mitomycin, MOZOBIL (Plerixafor), MUSTARGEN (Mechlorethamine Hydrochloride), MUTAMYCIN (Mitomycin C), MYLOSAR (Azacitidine), MYLOTARG (Gemtuzumab Ozogamicin), Nanoparticle Paclitaxel (Paclitaxel Albumin-stabilized Nanoparticle Formulation), NAVELBINE (Vinorelbine Tartrate), Nelarabine, NEOSAR (Cyclophosphamide), NEUPOGEN (Filgrastim), NEXAVAR (Sorafenib Tosylate), Nilotinib, NOLVADEX (Tamoxifen Citrate), NPLATE (Romiplostim), Ofatumumab, Omacetaxine Mepesuccinate, ONCASPAR (Pegaspargase), ONTAK (Denileukin Diftitox), Oxaliplatin, Paclitaxel, Paclitaxel Albumin-stabilized Nanoparticle Formulation, Palifermin, Palonosetron Hydrochloride, Pamidronate Disodium, Panitumumab, Pazopanib Hydrochloride, Pegaspargase, Peginterferon Alfa-2b, PEG-INTRON (Peginterferon Alfa-2b), Pemetrexed Disodium, Pertuzumab, PLATINOL (Cisplatin), PLATINOL-AQ (Cisplatin), Plerixafor, Pomalidomide, POMALYST (Pomalidomide), Ponatinib Hydrochloride, Pralatrexate, Predni sone, Procarbazine Hydrochloride, PROLEUKIN (Aldesleukin), PROLIA (Denosumab), PROMACTA (Eltrombopag Olamine), PROVENGE (Sipuleucel-T), PURINETHOL (Mercaptopurine), Radium 223 Dichloride, Raloxifene Hydrochloride, Rasburicas, Recombinant Interferon Alfa-2b, Regorafenib, REVLIMID (Lenalidomide), RHEUMATREX (Methotrexate), Rituximab, Romidepsin, Romiplostim, RUBIDOMYCIN (Daunorubicin Hydrochloride), Ruxolitinib Phosphate, Sipuleucel-T, Sorafenib Tosylate, SPRYCEL (Dasatinib), STIVARGA (Regorafenib), Sunitinib Malate, SUTENT (Sunitinib Malate), SYLATRON (Peginterferon Alfa-2b), SYNOVIR (Thalidomide), SYNRIBO (Omacetaxine Mepesuccinate), TAFINLAR (Dabrafenib), Tamoxifen Citrate, TARABINE PFS (Cytarabine), TARCEVA (Erlotinib Hydrochloride), TARGRETIN (Bexarotene), TASIGNA (Nilotinib), TAXOL (Paclitaxel), TAXOTERE (Docetaxel), TEMODAR (Temozolomide), Temozolomide, Temsirolimus, Thalidomide, TOPOSAR (Etoposide), Topotecan Hydrochloride, Toremifene, TORISEL (Temsirolimus), Tositumomab and I 131 Iodine Tositumomab, TOTECT (Dexrazoxane Hydrochloride), Trametinib, Trastuzumab, TREANDA (Bendamustine Hydrochloride), TRISENOX (Arsenic Trioxide), TYKERB (Lapatinib Ditosylate), Vandetanib, VECTIBIX (Panitumumab), VeIP, VELBAN (Vinblastine Sulfate), VELCADE (Bortezomib), VELSAR (Vinblastine Sulfate), Vemurafenib, VEPESID (Etoposide), VIADUR (Leuprolide Acetate), VIDAZA (Azacitidine), Vinblastine Sulfate, Vincristine Sulfate, Vinorelbine Tartrate, Vismodegib, VORAXAZE (Glucarpidase), Vorinostat, VOTRIENT (Pazopanib Hydrochloride), WELLCOVORIN (Leucovorin Calcium), XALKORI (Crizotinib), XELODA (Capecitabine), XGEVA (Denosumab), XOFIGO (Radium 223 Dichloride), XTANDI (Enzalutamide), YERVOY (Ipilimumab), ZALTRAP (Ziv-Aflibercept), ZELBORAF (Vemurafenib), ZEVALIN (Ibritumomab Tiuxetan), ZINECARD (Dexrazoxane Hydrochloride), Zoledronic Acid, ZOLINZA (Vorinostat), ZOMETA (Zoledronic Acid), and ZYTIGA (Abiraterone Acetate), including any formulation (e.g. liposomal, pegylated) any salt or any brand name of any generic agent included herein, including combinations thereof and/or in combination with any other active agent(s).

In embodiments, the invention may deliver cardiovascular drugs (for example, using any methods and/or compositions of the invention, including any of Aspects 1-92 above or any of Examples 1-14 below), including inhibitors of the renin-angiotensin system such as enalapril, lisinopril, ramipril, captopril, perindopril, trandolapril; angiotensin II receptors antagonists such as losartan; calcium channel blockers: nifedipine, amlodipine, nitrendipine, nimodipine, diltiazem, verapamil; simpathocolitic agents; adrenergic antagonists; atenolol, propanolol, nadolol, sotalol, timolol, metropolol, acebutolol, carvedilol; adrenergic agonists; prazosin, fentolamine; centrally acting agents such as methyldopa, clonidine, guanfacine, reserpine; direct arterial and venous vasodilators such as sodium nitroprusside, nitroglycerin, isosorbide 5-mononitrate, isosorbide dinitrate, amyl nitrite; antiarrythmic agents such as quinidine, procainamide, phenytoin, lidocaine, mexiletine, propafenone, flecainide, encainide, propranolol, acebutolol, amiodarone, sotalol, verapamil and diltiazem; digitalis; and cardiac glycosides such as digoxine, digitoxine, amrinone, and milrinone and combinations thereof and/or in combination with any other active agent(s).

In embodiments, the invention may deliver respiratory drugs, for example, using any methods and/or compositions of the invention, including any of Aspects 1-92 above or any of Examples 1-14 below, including respiratory drugs such as albuterol, levalbuterol, salmeterol, formoterol, fluticasone, budesonide, mometasone furoate, nedocromil, cromolyn sodium, omalizumab, zafirlukast, montelukast, zileuton, rloratidine, fexofenadine, cetirizine, epinephrine, doxapram, theophylline, progesterone, caffeine, colfosceril palmitate, beractant, calfactant, poractant alfa, pentamidine, tobramycin, ribavirin, zanamivir, guaifenesin, and varenicline and combinations thereof and/or with any other active agent(s).

In embodiments, the invention may deliver gastrointestinal drugs, for example, using any methods and/or compositions of the invention, including any of Aspects 1-92 above or any of Examples 1-14 below, including antacids (aluminum hydroxide, magnesium hydroxide, calcium carbonate, bismuth subsalicylate, sodium bicarbonate), proton pump inhibitors (omeprazole, lansoprazole, rabeprazole, esomeprazole, pantoprozole), histamine2 blockers (cimetidine, ranitidine hydrochloride, famotidine, nizatidine), and promotility agents (metoclopramide) and combinations thereof and/or with any other active agent(s).

In embodiments, the invention may deliver immunosuppresive drugs, for example, using any methods and/or compositions of the invention, including any of Aspects 1-92 above or any of Examples 1-14 below, such as immunosuppresive drugs including azathioprine, mycophenolate mofetil, cyclosporine, sandimmune (cyclosporine), methotrexate, leflunomide, cyclophosphamide, chlorambucil, and nitrogen mustard, and methotrexate and combinations thereof and/or with any other active agent(s).

In embodiments, the invention may deliver anti-inflammatory drugs, for example, using any methods and/or compositions of the invention, including any of Aspects 1-92 above or any of Examples 1-14 below, wherein the anti-inflammatory drugs include non-steroidal anti-inflammatory drugs (NSAIDS) (e.g., celecoxib, piroxicam, indomethacin, meloxicam, ketoprofen, sulindac, diflunisal), steroidal anti-inflammatory drugs (e.g., prednisone, cortisone, and methylprednisolone) and combinations thereof and/or with any other active agent(s).

In embodiments, the invention may deliver central nervous system (CNS) drugs, for example, using any methods and/or compositions of the invention, including any of Aspects 1-92 above or any of Examples 1-14 below, wherein the CNS drugs include hypnotics, anxiolytics, antipsychotics, barbituates, antidepressants, antiobesity, antihistamines, antiepileptics, antimanics, opioids, analgesics, anti-Parkinson, anti-Alzheimer's, anti-dementia, anti-substance dependence drugs, cannabinoids, 5HT-3 antagonists, monoamine oxidase inhibitors (MAOIs), selective serotonin reuptake inhibitors (SSRIs) and stimulants and combinations thereof and/or with any other active agent(s).

In embodiments, the invention may deliver analgesics, for example, using any methods and/or compositions of the invention, including any of Aspects 1-92 above or any of Examples 1-14 below, wherein the analgesics include analgesic combinations, antimigraine agents, CGRP inhibitors, cox-2 inhibitors, miscellaneous analgesics, narcotic analgesic combinations, narcotic analgesics, nonsteroidal anti-inflammatory drugs, salicylates, and the like. Anticonvulsants include AMPA receptor antagonists, barbiturate anticonvulsants, benzodiazepine anticonvulsants, carbamate anticonvulsants, carbonic anhydrase inhibitor anticonvulsants, dibenzazepine anticonvulsants, fatty acid derivative anticonvulsants, gamma-aminobutyric acid analogs, gamma-aminobutyric acid reuptake inhibitors, hydantoin anticonvulsants, oxazolidinedione anticonvulsants, pyrrolidine anticonvulsants, succinimide anticonvulsants, triazine anticonvulsants, and the like; Antiemetic/antivertigo agents include 5HT3 receptor antagonists, anticholinergic antiemetics, NK1 receptor antagonists, phenothiazine antiemetics, and the like. Antiparkinson agents include anticholinergic antiparkinson agents, dopaminergic antiparkinson agents, and the like. Anxiolytics, sedatives, and hypnotics include barbiturates, benzodiazepines, cholinergic agonists, and the like. Antidepressants include SSRIs (e.g., fluoxetine, sertraline, paroxetine), SNRIs (e.g. desvenlafaxine, duloxetine, venlafaxine), MAOIs (e.g. rasagiline, selegiline, isocarboxazid) and TCAs (e.g. amitriptyline, amoxapine, desipramine, doxepin) and the like. Antipsychotics include typical antipsychotics (e.g. haloperidol) and atypical antipsychotics (e.g. aripiprazole) and combinations thereof and/or with any other active agent(s).

In embodiments, the invention may deliver ophthalmic drugs such as pilocarpine and cyclopentolate, and combinations thereof and/or with any other active agent(s), for example, using any methods and/or compositions of the invention, including any of Aspects 1-92 above or any of Examples 1-14 below.

In embodiments, the invention may deliver otolaryngology drugs such as fluticasone, miconazole, and nystatin, and combinations thereof and/or with any other active agent(s), for example, using any methods and/or compositions of the invention, including any of Aspects 1-92 above or any of Examples 1-14 below.

Additional drugs which can be included in compositions and methods of the invention as well as diseases and conditions which can be treated can be found in publicly available drug references (see Remington, The Science and Practice of Pharmacy. Easton, Pa.: Mack Pub. Co., 1995; Goodman, L. S., Brunton, L. L., Chabner, B., & Knollmann, B. C. (2011). Goodman & Gilman's pharmacological basis of therapeutics. New York: McGraw-Hill; O'Neil, M. J. (2006). The Merck index: An encyclopedia of chemicals, drugs, and biologicals. Whitehouse Station, N.J: Merck; Center for Drug Evaluation and Research (U.S.)., & Center for Drug Evaluation and Research (U.S.). (1985). Orange book: Approved drug products with therapeutic equivalence evaluations. Rockville, Md.: U.S. Dept. of Health and Human Services, Food and Drug Administration, Center for Drug Evaluation and Research, Office of Pharmaceutical Science, Office of Generic Drugs); these and any edition available at the time of this disclosure are hereby incorporated by reference herein in their entireties.

In embodiments, the invention may deliver a macromolecule which can be used in methods and/or compositions of the invention, including in any of Aspects 1-92 above or in any of the Examples 1-14 below and, for example may include an antibody, antibody fragment, antibody-drug conjugate, peptide, peptide-drug conjugate, protein, polypeptide, fusion protein, multivalent binding protein, blood and blood product, nucleic acid, nucleotide, oligonucleotide, antisense oligonucleotide, short interfering RNA (siRNA), micro-interfering RNA (miRNA); small, temporal RNA (stRNA); short, hairpin RNA (shRNA), aptamer, ribozyme, viral vector (e.g. adenovirus, adeno-associated virus (AAV), retrovirus, and lentivirus), plasmid, cells and/or tissues (autologous, allogeneic, or xenogeneic), which can include liver cells/tissues, kidney cells/tissues, neurons, primary cells, immortalized cells, any cell of the immune system, pancreatic cells/tissues (e.g. islets, beta cells), muscle cells, stem cells (multipotent, pluripotent, totipotent), and so on. Examples of biologics products include adalimumab (Humira), rituximab (Rituxan), etanercept (Enbrel), trastuzumab (Herceptin), bevacizumab (Avastin), infliximab (Remicade), insulin glargine (Lantus), pegfilgrastim (Neulasta), interferon beta-1a (Avonex), ranibizumab (Lucentis), and epoetin alfa (Epogen).

Additional biologics that can be used in methods of the invention and/or compositions of the invention, including in any of Aspects 1-92 above or in any of the Examples 1-14 below, can be found in the U.S. Food and Drug Administration's Purple Book, which is publicly available on the U.S. FDA website and hereby incorporated by reference.

Any active agent, including those recited in this application can be delivered using any methods and/or compositions of the invention, including any of Aspects 1-92 above or any of Examples 1-14 below. Active agents can be used alone or together and in any and all combinations. For example, any analgesic can be administered with any anti-inflammatory and/or in combination with any antibacterial, antifungal or antiviral, including those specifically listed in this application. To this end, each of the particular active agents identified herein can be used in combination with any other active agent(s) disclosed herein as well.

The following Examples are illustrative and should not be interpreted as limiting.

### Example 1

A poly(N-isopropyl-acrylamine) hydrogel is synthesized and embedded with near infrared (NIR)-absorbing nanostructures (e.g., nanorods, nanoshells, and carbon nanotubes). The thermo-sensitive hydrogel is also loaded with antibiotic. The hydrogel is implanted into the vas deferens and occludes sperm for effective contraception. Upon exposure to NIR light above the scrotal skin, the antibiotic is released from the hydrogel. The same hydrogel may also be implanted into the fallopian tubes for female contraception, or in the subcutaneous space, where the gel serves as a drug depot.

### Example 2

An in situ or injectable hydrogel system that has a suspension of micronized testosterone, such that upon injection the micronized testosterone is encapsulated within the hydrogel. For example, an in situ gelling via bioorthogonal crosslinking of PEG hydrogel that encapsulates a suspension micronized testosterone upon gelling that is placed into the subcutaneous space. The gel delivers testosterone for 4 months and then degrades. The size and properties of degradation products are such that they are excreted out. This system allows regular administration of a long-term hormonal delivery system.

### Example 3

An in situ or injectable hydrogel system that has a suspension of testosterone loaded polymeric microparticles, such that upon injection the testosterone loaded particles are encapsulated within the hydrogel. For example, an in situ gelling via bioorthogonal crosslinking of PEG hydrogel where testosterone is encapsulated in a polymeric microparticle that is entrapped within the hydrogel upon injection into the subcutaneous space. The gel delivers testosterone for 6 months and then degrades. The size and properties of degradation products are such that they are excreted out. This system allows regular administration of a long-term hormonal delivery system.

### Example 4

An in situ or injectable hydrogel that is loaded with a suspension of anti-HIV drugs/anti-virals or drug delivery vehicles (such as those listed above) loaded with the drug/antivirals. For example, an in situ gelling via bioorthogonal crosslinking of PEG hydrogel loaded with emtricitabine and tenofovir disoproxil fumarate that is implanted within the vasa deferentia. The hydrogel occludes the vessel resulting in infertility as well as prevents HIV infection for the lifetime of the implant. The lifetime of the implant is dependent upon the formulation which for example would last 6 months, 12 months, 18 months, or 24 months.

### Example 5

An in situ or injectable hydrogel that is loaded with a suspension of anti-HIV drugs/anti-virals or drug delivery vehicles (such as those listed above) loaded with the drugs/antivirals. For example, an in situ gelling via bioorthogonal crosslinking of PEG hydrogel loaded with emtricitabine and tenofovir disoproxil fumarate that is implanted within the fallopian tubes. The hydrogel occludes the tubes resulting in infertility as well as prevents HIV infection for the lifetime of the implant. The lifetime of the implant is dependent upon the formulation which for example would last 6 months, 12 months, 18 months, or 24 months.

### Example 6

An in situ or injectable hydrogel that is loaded with a suspension of antibiotics/anti-virals to prevent sexually transmitted infections or drug delivery vehicles (such as those listed above) loaded with the drugs/antivirals. For example, an in situ gelling via bioorthogonal crosslinking of PEG hydrogel loaded with soluble doxycycline that is implanted within the vasa deferentia. The hydrogel occludes the vessel resulting in infertility as well as prevents chlamydia, gonorrhea, and/or syphilis infection for the lifetime of the implant. The lifetime of the implant is dependent upon the formulation which for example would last 6, 12, 18, or 24 months.

### Example 7

An in situ or injectable hydrogel that is loaded with a suspension of antibiotics/anti-virals to prevent sexually transmitted infections or drug delivery vehicles (such as those listed above) loaded with the drugs/antivirals. For example, an in situ gelling via bioorthogonal crosslinking of PEG hydrogel loaded with soluble doxycycline that is implanted within the fallopian tubes. The hydrogel occludes the tubes resulting in infertility as well as prevents chlamydia, gonorrhea, and/or syphilis infection for the lifetime of the implant. The lifetime of the implant is dependent upon the formulation which for example would last 6, 12, 18, or 24 months.

### Example 8

An in situ or injectable hydrogel that is loaded with a suspension of antibiotics/anti-virals to prevent sexually transmitted infections or drug delivery vehicles (such as those listed above) loaded with the drugs/antivirals. For example, an in situ gelling via bioorthogonal crosslinking of PEG hydrogel loaded with a suspension of nanocrystals of doxycycline that is implanted within the vasa deferentia. The hydrogel occludes the vessel resulting in infertility as well as prevents chlamydia, gonorrhea, and/or syphilis infection. The lifetime of the implant is dependent upon the formulation which for example would last 6, 12, 18, or 24 months.

### Example 9

An in situ or injectable hydrogel that is loaded with a suspension of antibiotics/anti-virals to prevent sexually transmitted infections or drug delivery vehicles (such as those listed above) loaded with the drugs/antivirals. For example, an in situ gelling via bioorthogonal crosslinking of PEG hydrogel loaded with a suspension of nanocrystals of doxycycline that is implanted within the fallopian tubes. The hydrogel occludes the tubes resulting in infertility as well as prevents chlamydia, gonorrhea, and/or syphilis infection for the lifetime of the implant. The lifetime of the implant is dependent upon the formulation which for example would last either 6 months, 12 months, 18 months, or 24 months.

### Example 10: Cumulative Release of Free Doxycycline (× mg/mL) from a Hydrogel

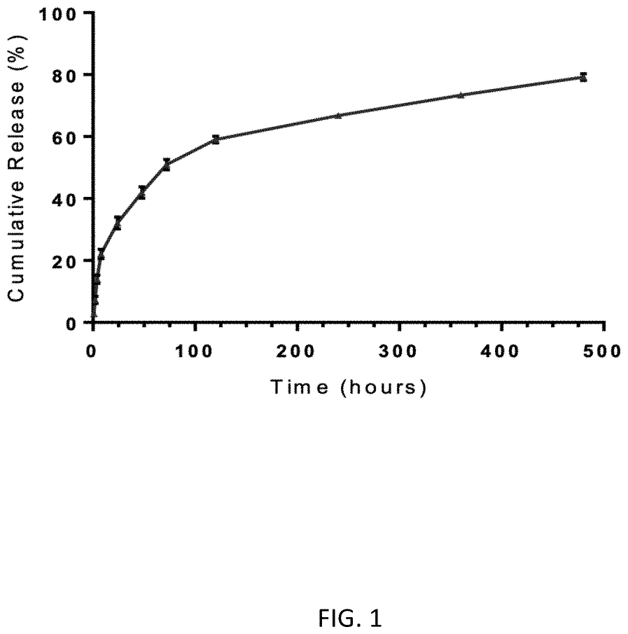

According to an embodiment of the invention, a hydrogel is loaded with free doxycycline. The hydrogel exhibits 50.96% cumulative release at 72 hours and 79.22% cumulative release at 480 hours (20 days) (FIG. 1).

### Example 11: Cumulative Release of Free Doxycycline (5× mg/mL) from Hydrogel

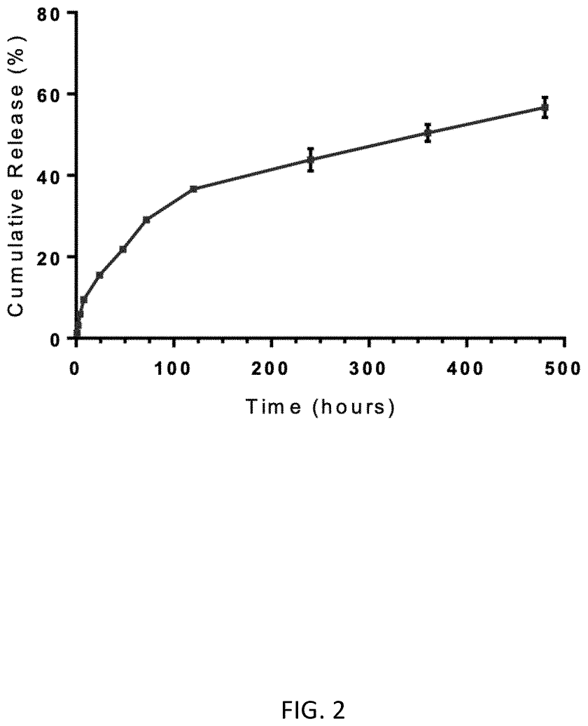

According to an embodiment of the invention, a hydrogel is loaded with free doxycycline. The hydrogel exhibits 50.43% cumulative release of drug at 360 hours (15 days) and 56.70% at 480 hours (20 days) (FIG. 2).

### Example 12: Generation of Nanoparticle Emulsion W/O/W

For particles prepared using the double emulsion solvent evaporation method, the polymers will be dissolved in dichloromethane at ˜5% w/v of polymer. To load a water-soluble compound, A W/O emulsion consisting of 100 μL of aqueous API solution including PVA surfactant and 1 mL of the polymer solution will be prepared by probe sonication for 1-3 min. This emulsion (1.1 mL) will be subsequently added to 10 mL of an external aqueous phase containing 1-5% w/v PVA in water. The samples will be further sonicated for 2-5 min to form a W/O/W emulsion. The particles will be hardened by evaporation of the organic solvent under stirring at room temperature for 2-24 h. Subsequently, they will be purified and isolated as detailed below.

After generation or modification of the particles, they will be isolated and purified as follows. Particles of large size will be removed by filtration through 1.0, 0.8, 0.45, or 0.22 μm filter (Whatmann) depending on the size range of particles desired, respectively. If an intermediate size range is desired, the particles will be put through a filter with a larger size and collected on top of a filter of smaller size (i.e. particles of ˜300 nm can be collected by passing through a 0.45 μm and collecting on top of a 0.22 μm filter). The particles will be washed and concentrated by tangential flow filtration (TFF, Pall) with MWCO of 100 kDa to remove surfactants, salts, and unencapsulated components with multiple washes in deionized (DI) water. Subsequently, they will be collected by centrifugation at 17,000 RPM, 4° C., 30 minutes with multiple washes. Larger quantities can be processed in the Large-Centrifuge (Beckman-Coulter Avanti J30I) which can ultracentrifuge up to 800 ml in a single run. Collected NP pellet will be lyophilized by freeze drying (Harvestright freeze dryer). The samples will be analyzed using GPC-4D. The GPC-4D will provide values as seen in Table 2 below.

TABLE 2
List of GPC-4D values.

| Value | Description |
|---|---|
| MHS Intercept (K) | Mark-Houwink constant “K” |
| MHS slope (a) | Mark-Houwink constant alpha |
| Mₙ (kDa) | Number average Molecular weight |
| Mₚ (kDa) | Peak molecular weight |
| Mᵥ (kDa) | Viscosity average molecular weight |
| M_{w} (kDa) | Weight average molecular weight |
| M_{z} (kDa) | Z-average molecular weight |
| Polydispersity | Distribution of molecular mass |
| (M_{w}/Mₙ) | |
| rₙ (nm) | Number-average mean square radius |
| r_{w} (nm) | Weight-average mean square radius |
| r_{z} (nm) | Z average radius |
| r_{avg} (nm) | Average mean square radius |
| rₕₒ ((nm) | Number-average hydrodynamic radius |
| r_{hw} (nm) | Weight-avg mean hydrodynamic radius |
| r_{hz} (nm) | Z-average hydrodynamic radius |
| R_{h,avg} | Average hydrodynamic radius |
| [η]ₙ (mL/g) | Number-average intrinsic viscosity |
| [η]_{w} (mL/g) | Weight-average intrinsic viscosity |
| [η]_{z} (mL/g) | Z-average intrinsic viscosity |
| dn/dc | Refractive index increment from batch- |
| | mode analysis |

### Example 13: Microparticle Generation

PLGA (of preselected properties as required by project) will be dissolved in dichloromethane (DCM) at 17-20% w/v and then pipetted into a 50× volume excess of aqueous 0.5-1.0% PVA (31 kDa, 88% hydrolyzed) with stirring at 700 RPM overnight. The microparticles will be sized by filtration to collect particles between appropriate sizes of mesh. For instance, passing particles through 150 μm nylon mesh (Component supply) and collecting them on top of a 7 μm mesh (Component supply). The subsequently collected particles will be washed with water and lyophilized (Harvestright Freeze dryer). API will either be introduced directly into the DCM solution (hydrophobic API) or incorporated as a double-emulsion for hydrophilic API.

For the delivery of doxycycline, in general, the incorporation of free base (monohydrate) is preferential as it has much easier incorporation into PLGA as well as reduced dissolution into water, which will increase release longevity. Doxycycline has a strong absorbance peak ˜375 nm which makes analysis by UV-V is feasible. If Doxycycline monohydrate can be dissolved directly into the DCM phase, potentially with the assistance of ethanol, then there may be no need for double-emulsion loading at all.

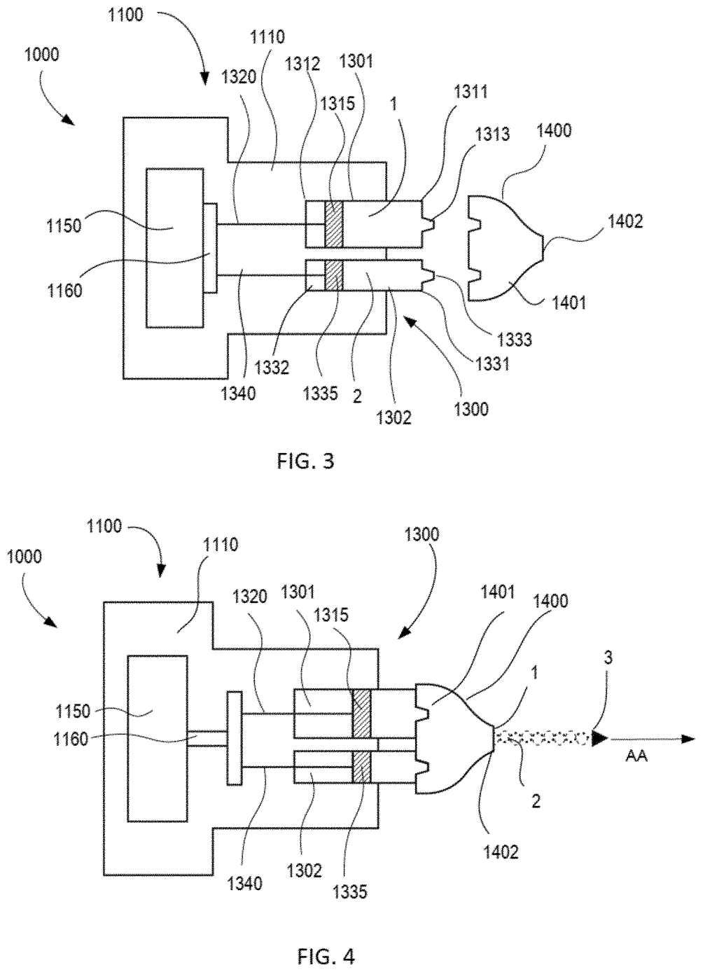

Any of the compositions and methods described herein can be used with any suitable delivery devices or systems. For example, FIGS. 3 and 4 are schematic illustrations of a portion of a system 1000 (also referred to as a delivery system) according to an embodiment. As described herein, the system 1000 is configured to convey and combine multiple biomaterial components that form a biomaterial product that is delivered to a target location. The delivery device 1100 includes a housing 1110 and a drive assembly 1150. The housing 1110 is configured to receive at least a portion of the container assembly 1300. The housing 1110 can also contain the drive assembly 1150. The housing 1110 can be made from any suitable material or materials and can provide any suitable structural components to receive and/or retain the portion of the container assembly 1300 and perform any of the functions described herein. For example, in some embodiments, the housing 1110 can be constructed from multiple components that are joined together (e.g., via a hinged joint, a mechanical fastener or the like) to surround and/or secure the container assembly 1300. In some embodiments, for example, the housing 1100 (or any of the housings described herein) can include a movable lid or cover that can reveal a container portion within which the container assembly 1300 can be removably coupled. In use, the lid or cover can be closed to secure the container assembly 1300 within the housing 1110. In some embodiments, the housing 1100 (or any of the housings described herein) can include a lock member (or set of lock members) that retain the container assembly 1300 within the housing and can prevent premature and/or undesired removal of the container assembly 1300.

The drive assembly 1150 can be any suitable assembly or mechanism that produces a drive force to convey the first biomaterial component 1 (also referred to as the first component), or the second biomaterial component 2 (also referred to as the second component), or both the first component 1 and the second component 2 from the container assembly 1300 as described herein. More specifically, the drive assembly 1150 can produce the drive force and/or convey the components within a desired velocity range, force range, and/or range of flow rates. By controlling the delivery characteristics of the first component 1, the second component 2, and/or the delivered product 3, the drive assembly 1150 can repeatably deliver an accurate amount of the delivered product 3 to the target location. This, in turn, can lead to more consistent and improved outcomes. Additionally, where the first component 1 and/or the second component 2 includes a therapeutic agent, controlled delivery prevents damage of the therapeutic agent as it is being conveyed to the target location. For example, if the therapeutic agent includes a biologic, controlled delivery prevents shearing of the biologic as it is conveyed from the container assembly 1300 to the target location. In some embodiments, the therapeutic agent is at least one of a small molecule, a biologic, an antibiotic and/or an anti-viral.

Controlling the delivery characteristics can also ensure that any desired reactions between the first component 1 and the second component 2 (e.g., a cross-linking reaction) are completed within the system 1000. Said another way, controlling the delivery characteristics can ensure that the delivered product 3 is fully formed within the system (e.g., the delivery member 1500), thereby ensuring that that the first component 1 and the second component 2 are not delivered while the product 3 is still yet to be formed (or is only partially formed). Controlling the delivery characteristics can also limit potential damage to the target tissue. Such damage can be caused by delivering an improper amount of the product or delivering the product too fast or at a force that causes tissue damage. Finally, controlling the delivery characteristics can also limit clogging or blockage within the system 1000 (e.g., the delivery member 1500). In other embodiments, it may be beneficial to deliver the product 3 partially formed as it exits the system 1000.

As shown, the drive assembly 1150 includes a drive member 1160 that is operably coupled to the container assembly 1300 such that, upon actuation, the drive assembly 1150 can convey the first component 1 and the second component 2 from the container assembly 1300. The drive assembly 1150 can include any suitable mechanism for producing the drive force. For example, in some embodiments, the drive assembly can include an electromechanical driver (not shown in FIGS. 3 and 4) to produce the drive force. Such electromechanical drivers can include, for example, a motor-driven linear actuator, a hydraulic actuator (e.g., that includes a pump driven by an electronic component), a magnetic-based actuator, a pneumatic actuator that includes an electromechanical valve to control a pressure applied to the drive member 1160, or any other suitable electromechanical driver of the types described herein. In some embodiments, the drive assembly 1150 and/or the delivery device 1100 can include an electronic control system (not shown) that controls the electromechanical driver and any other aspect of the drive assembly to control the delivery characteristics of the first component 1, the second component 2, and/or the delivered product 3, as described herein. Although the system 1000 is shown and described as including the drive assembly 1150 that produces a drive force, in some embodiments the drive assembly 1150 can be manually operated to supply the drive force.

The container assembly 1300 includes a first container 1301 and a second container 1302, and can be coupled to and/or received within the housing 1110. The first container 1301 has a first end portion 1311, a second end portion 1312, and includes an elastomeric member (or stopper) 1315 therein. The first container 1301 defines a volume that is bounded on one side by the elastomeric member 1315 and that contains a first component 1. The first container 1301 includes a first plunger 1320 having an end portion movably disposed within the first container 1301 such that movement of the first plunger 1320 will cause movement of the elastomeric member 1315 to convey the first component 1 from the first container 1301. The opposite end of the first plunger 1320 is operably coupled to (e.g., is configured to engage) the drive member 1160. The second container 1302 has a first end portion 1331, a second end portion 1332, and includes an elastomeric member (or stopper) 1335 therein. The second container 1302 defines a volume that is bounded on one side by the elastomeric member 1335 and that contains a second component 2. The second container 1302 includes a second plunger 1340 having an end portion movably disposed within the second container 1302 such that movement of the second plunger 1340 will cause movement of the elastomeric member 1335 to convey the second component 2 from the second container 1302. The opposite end of the second plunger 1340 is operably coupled to (e.g., is configured to engage) the drive member 1160. In some embodiments, the elastomeric member is made of a butyl rubber such as chlorobutyl or bromobutyl. In some embodiments, the elastomeric member can be coated with a film or other coatings such as ethylene tetrafluoroethylene (ETFE) or fluorinated ethylene propylene (FEP).

The first container 1301 and the second container 1302 (and any of the containers described herein) can be any suitable containers. For example, the first container 1301 and/or the second container 1302 can be a cartridge, an ampule, or a syringe. Moreover, the first container 1301 and the second container 1302 (and any of the containers described herein) can be of any suitable size and can be constructed from any suitable material such a type I borosilicate glass. For example, in some embodiments, the first container 1301 and the second container 1302 can have different sizes (e.g., different diameters). In this manner, the container assembly can accommodate delivering different volumes of the first component 1 and the second component 2 while maintaining a constant stroke length. In other embodiments, the first container 1301 and the second container 1302 can be the same size.

The first component 1 and the second component 2 can be any of the biomaterial components described herein. By way of example, in some embodiments, the first component 1 and the second component 2 can each be a water soluble component (e.g., monomer, macromer, polymer, or the like) that is capable of crosslinking (e.g., with the other component) to form a hydrogel (as the delivered biomaterial product).

The first container 1301 and the second container 1302 are configured to be coupled to the connector 1400. By having the containers as separate articles from the connector, the first container 1301 and the second container 1302 can be commercially available containers (e.g., syringes) within which the first component 1 and the second component 2, respectively, can be prepared for use. Moreover, this arrangement allows the first component 1 to be prepared within the first container 1301 (e.g., via mixing, dilution, etc.) separately from when the second component 2 is prepared within the second container 1302. In other embodiments, however, the container assembly 1300 can include a first container and a second container that are integrally and/or monolithically constructed with the connector. In yet other embodiments, the container assembly 1300 can include a single container that contains both the first component 1 and the second component 2.

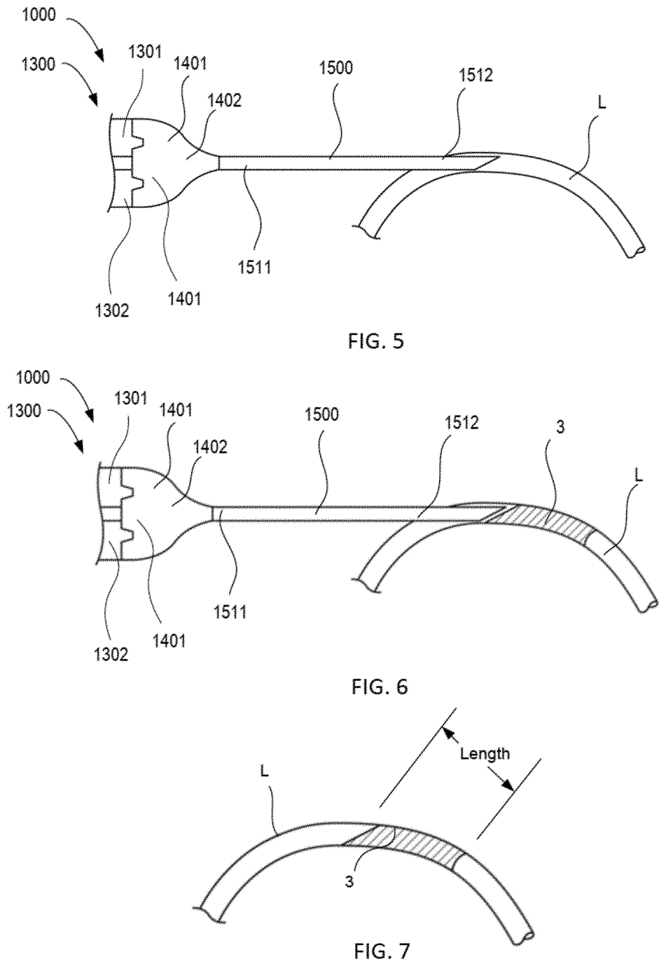

As shown, the connector 1400 includes a first (or input) end portion 1401 and a second (or output) end portion 1402. The first end portion 1401 is configured to receive a tip (or connector) 1313 of the first container 1301 and a tip (or connector) 1333 of the second container 1302. The second end portion 1402 is configured to be coupled to a delivery member 1500 (see e.g., FIGS. 5 and 6). In this manner, the first component 1 can be conveyed from the first container 1301, into the first end portion 1401 of the connector 1400, and out of the second end portion 1402 of the connector to the delivery member 1500. Similarly, the second component 2 can be conveyed from the second container 1302, into the first end portion 1401 of the connector 1400, and out of the second end portion 1402 of the connector 1400 to the delivery member 1500. In some embodiments, the connector 1400 can be a mixing connector within which the first component 1 is mixed with the second component 2 before the two components are conveyed into the delivery member 1500. In other embodiments, however, the connector 1400 can maintain the first component 1 separate from the second component 2, and the two components are conveyed into and mixed within the delivery member 1500. By maintaining separate flow paths within the connector 1400, the reaction (e.g., crosslinking) between the first component 1 and the second component 2 can be performed outside of the connector 1400 (i.e., within the delivery member 1500), thereby limiting the likelihood of clogging with the connector 1400. In this manner, the connector 1400 can be used for multiple injections.

The delivery member 1500 can be any suitable delivery member, such as a needle, a catheter, or any other device through which the first component 1, the second component 2, and/or the biomaterial product 3 can be delivered to the target location. In some embodiments, the connector 1400 and the delivery member 1500 can be monolithically constructed or otherwise pre-assembled prior to use. In other embodiments, the connector 1400 can be separate from the delivery member 1500 and coupled to the delivery member 1500 as a part of the delivery procedure.

In use, after the container assembly 1300 is prepared and coupled to the delivery device 1100, the drive assembly 1150 can be actuated to produce the drive force. In this manner, the drive assembly 1150 (and the drive member 1160) can move the first plunger 1320 and the second plunger 1340 simultaneously for a time period to dispense a portion of the first component 1 from the first container 1301 and a portion of the second component 2 from the second container 1302. The first component 1 and the second component 2 are conveyed through the connector 1400, as shown by the arrow AA in FIG. 4. As described above, the first component 1 and the second component 2 can react (e.g., within the delivery member 1500, not shown in FIG. 4) to form the biomaterial product 3. The drive assembly 1150 is configured to move the first plunger 1320 and the second plunger 1340 such that the first component 1 and the second component 2 exit the connector 1400 at an exit velocity to induce crosslinking of the first component 1 and the second component 2 within the delivery member 1500 and/or as the first component 1 and the second component 2 exit the delivery member 1500.

As described herein, the first component 1 and the second component 2 are formulated to have an initial storage modulus (initial G′) and an initial loss modulus (initial G″) when initially combined such that a ratio of the initial G″ to the initial G′ is between about 5 and about 100. In some embodiments, the drive assembly 1150 is configured to move the first plunger 1320 and the second plunger 1340 to combine the first component 1 and the second component 2 to achieve the initial G″ to the initial G′ of between about 5 and about 100. In some embodiments, the gelation time (also referred to herein as gelation rate) after the first component 1 and the second component 2 are combined is less than 120 seconds. In some embodiments, the gelation time is between 1 and 60 seconds.

The system 1000 (and any of the systems described herein) can be used to deliver a biomaterial product (such as any of the hydrogels described herein) to a target location. For example, FIGS. 5-7 are schematic illustrations showing the system 1000 being used to deliver a biomaterial product 3 to a body part, organ, duct, cavity/space or lumen L. In use, the container assembly 1300 can be readied for use by preparing (e.g., mixing, reconstituting, etc.) and loading the first component 1 into the first container 1301 and the second component 2 into the second container 1302. The container assembly 1300 can then be coupled to (or loaded into) the delivery device 1100 and primed for use, in accordance with any of the methods described herein. The delivery member 1500 is then inserted into the body part, organ, duct, cavity/space or lumen L, as shown in FIG. 5. In some embodiments, the delivery member 1500 can be inserted before being connected to the connector 1400. In other embodiments, however, the delivery member 1500 can be coupled to the connector 1400 and then inserted into the body part, organ, duct, cavity/space or lumen L. As shown in FIG. 6, the delivery device 1100 can be actuated to initiate delivery of the first component 1 and the second component 2 through the connector 1400 and the delivery member 1500, as described above. Specifically, the delivery device 1100 can convey the first component 1 and the second component 2 for a delivery time period and within a desired velocity range. In this manner, the desired volume or length of the biomaterial product 3 can be delivered into the body part, organ, duct, cavity/space or lumen L. The delivery member 1500 can then be removed from the body part, organ, duct, cavity/space or lumen L, as shown in FIG. 7. The body part, organ, duct, cavity/space or lumen L can be any suitable body part, organ, duct, cavity/space or lumen, such as, for example, an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

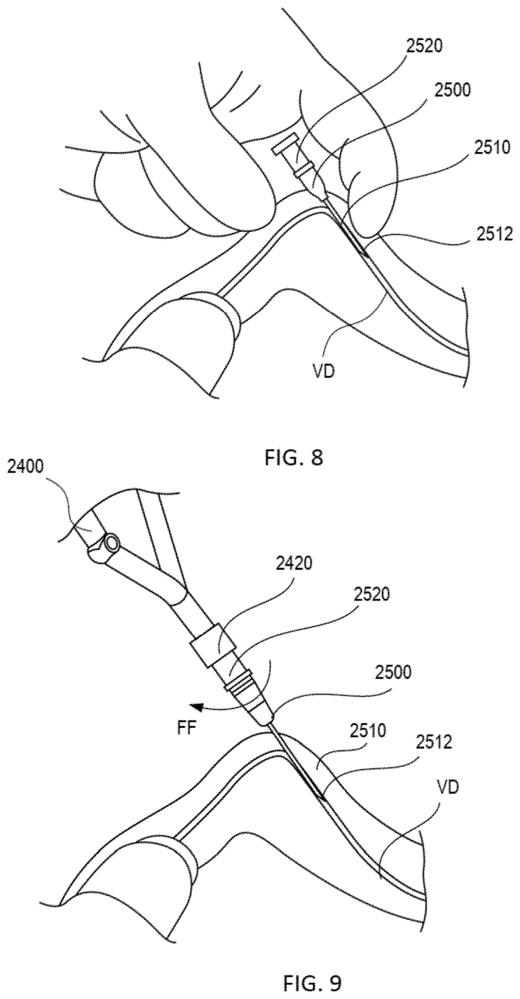

In some embodiments, any of the compositions described herein can be delivered with any suitable delivery devices or systems, such as the delivery system 1000. Referring to FIG. 8, any of the compositions, methods, or delivery systems described herein can be delivered to a target location via a first delivery member 2500 inserted into the body part, organ, duct, cavity/space or lumen with the target location. The first delivery member 2500 can include an end portion 2512 that is inserted into a first vas deferens VD of a patient. After the first delivery member 2500 is inserted, the first delivery member 2500 can be coupled to a container assembly, such as the container assembly 1300 described herein. In some embodiments, the system can be primed before the first delivery member 2500 is coupled to the container assembly. Referring to FIG. 9, the first delivery member 2500 can be coupled to the container assembly via the connector 2400. The first delivery member 2500 can be coupled to the connector 2400 by rotating at least one of the fitting 2420 or the delivery member 2500 relative to the other.

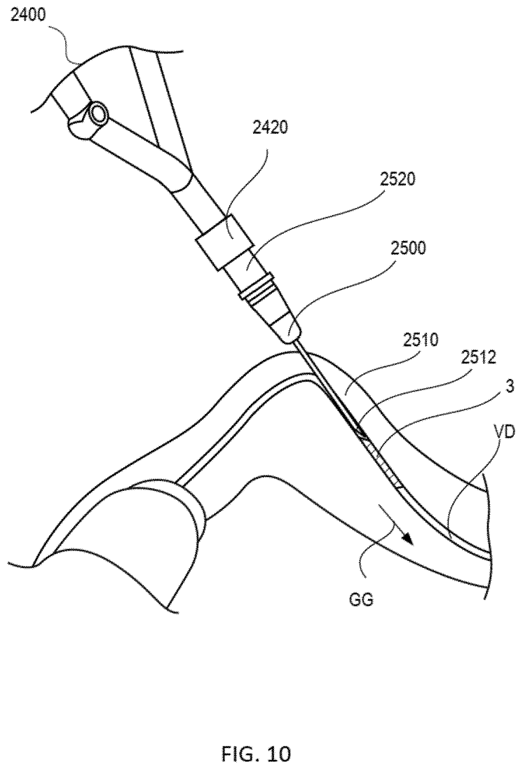

The delivery device, such as the delivery system 1000, can be actuated to cause the drive assembly to produce a first drive force to convey a first portion of the first component and a first portion of the second component from the container assembly and through the first delivery member 2500. The first component crosslinks with the second component to form a first hydrogel within the first delivery member. The continued conveying causes the first hydrogel to be conveyed into the first body part, organ, duct, cavity/space or lumen. Referring to FIG. 10, the first hydrogel (identified as the biomaterial product 3) is conveyed into the vas deferens VD as shown by the arrow GG. The delivery can be controlled as described herein to produce a desired volume and/or length of the first hydrogel within the vas deferens.

After the first actuating, the first delivery member 2500 is decoupled from the container assembly. The first delivery member can optionally be removed and discarded. A second delivery member is inserted into a second body part, organ, duct, cavity/space or lumen. The second delivery member can be the delivery member 2500 and the inserting can include inserting the end portion 2512 into a second vas deferens VD of a patient. After the second delivery member is inserted, the second delivery member coupled to the container assembly.

The delivery device is actuated at second time to cause the drive assembly to produce a second drive force to convey a second portion of the first component and a second portion of the second component from the container assembly and through the second delivery member. The first component crosslinks with the second component to form a second hydrogel within the second delivery member. The continued conveying causes the second hydrogel to be conveyed into the second body part, organ, duct, cavity/space or lumen.

Although in many instances it can be undesirable to deliver any substance other than the therapeutic material into the body, the systems described herein can be used to advantageously prime (or prepare) a vessel within which the biomaterial product is to be delivered. Similarly stated, in some embodiments, a method can include delivering a priming fluid (e.g., air, saline, or any other suitable inert fluid) into the target vessel before delivering the biomaterial product. Delivery of a priming fluid can prepare the body part, organ, duct, cavity/space or lumen by dilating (or enlarging) the body part, organ, duct, cavity/space or lumen, washing away impurities, and/or by producing a coating on the walls that can improve the efficacy of the delivered biomaterial product and aid in proper placement of the biomaterial product.

The present invention has been described with reference to particular embodiments having various features. In light of the disclosure provided above and the claims provided below, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range, to the tenth of the unit disclosed, is also specifically disclosed. Any smaller range within the ranges disclosed or that can be derived from other endpoints disclosed are also specifically disclosed themselves. The upper and lower limits of disclosed ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A composition, comprising:
a first component and a second component, the first component formulated to be crosslinked with the second component to form a hydrogel;
wherein the first component is a multi-arm polyethylene glycol terminated with a thiol and the second component is a multi-arm polyethylene glycol terminated with a maleimide;
the first component and the second component being formulated to have an initial storage modulus (initial G′) and an initial loss modulus (initial G″) when the first component and the second component are initially combined, a ratio of the initial G″ to the initial G′ being between about 5 and about 100; and
the first component and the second component being formulated to have a gelation storage modulus (gelation G′) and a gelation loss modulus (gelation G″) at a gelation time after the first component and the second component are combined, a ratio of the gelation G″ to the gelation G′ being less than about 1, the gelation time being less than about 120 seconds.

2. The composition of claim 1, wherein:
the hydrogel further comprises one or more therapeutic agent(s); and
the hydrogel is configured to release the therapeutic agent(s) to a patient over a period of at least 5 days, or at least a week, or at least a month.

3. The composition of claim 1, further comprising one or more therapeutic agent(s), wherein the therapeutic agent(s) are chosen from one or more of hormones, anti-HIV drugs, antibiotics, and/or anti-virals.

4. The composition of claim 1, wherein the gelation time is less than about 60 seconds.

5. (canceled)

6. The composition of claim 1, wherein the hydrogel is capable of being disposed within one or more body part, organ, duct, cavity/space or lumen chosen from:
an artery, vein, capillary, vessel, tissue, intra-organ space, lymphatic vessel, a femoral artery, popliteal artery, coronary and/or carotid artery, esophagus, cavity, nasopharyngeal cavity, ear canal, tympanic cavity, sinus, sinuses of the brain, any artery of the arterial system, any vein of the venous system, heart, larynx, trachea, bronchi, stomach, duodenum, ileum, colon, rectum, bladder, kidney, ureter, ejaculatory duct, epididymis, vas deferens, urethra, uterine cavity, vaginal canal, fallopian tube, cervix, duct, bile duct, a hepatic duct, a cystic duct, a pancreatic duct, a parotid duct, organ, a uterus, prostate, organ of the gastrointestinal tract, organ of the circulatory system, organ of the respiratory system, organ of the nervous system, urological organ, subcutaneous space, intramuscular space, or interstitial space.

7. The composition of claim 6, wherein the hydrogel is capable of occluding the body part, organ, duct, cavity/space or lumen, in whole or in part, in a manner to:
cause infertility; and/or
prevent or treat one or more infection or disease, sexually transmitted infection or disease, or HIV infection, for up to 1 week, 1 month, 3 months, 6 months, 12 months, 18 months, or 24 months.

8. The composition of claim 1, wherein at least one of the first component and/or the second component are dissolved in a solvent and have a weight percentage within the solvent of between about 1 wt % and 30 wt %.

9. The composition of claim 1, wherein at least one of the first component and/or the second component are dissolved in a solvent chosen from any one or more of:
Acetic Acid-Sodium Acetate (AA),
Citric Acid-Sodium Citrate (CA),
Citric Acid (0.2 M)-Phosphate Buffer (0.1 M) (CP), or
Phosphate Buffer (PB).

10. The composition of claim 9, wherein the first and/or second component comprises any one or more functional group chosen from Thiol (SH), Maleimide (MAL), o-nitrobenzyl (ONB), Hydrazide (HZ), Isocyanate (IC), Amine (NH), Succinimidyl Glutaraldehyde (SG), Aldehyde (AD), or Epoxide (EP).

11-53. (canceled)

54. The composition of claim 8, wherein at least one of the first component and/or the second component are dissolved in a solvent and have a weight percentage within the solvent of between about 1-25 wt %.

55. The composition of claim 1, further comprising one or more therapeutic agent(s).

56. The composition of claim 55, wherein the hydrogel is capable of releasing the therapeutic agent(s) over a period of time of at least 5 days.

57. The composition of claim 55, wherein one or more of the therapeutic agents has pharmacological activity against one or more sexually transmitted diseases and/or provides for male and/or female contraception.

58. The composition of claim 55, wherein:
the first component comprises one or more of the therapeutic agent(s); or
the second component comprises one or more of the therapeutic agent(s); or
the first and second components comprise one or more of the therapeutic agent(s); or
the first component comprises a first therapeutic agent and the second component comprises a second therapeutic agent that is the same or different as the first therapeutic agent.

59. The composition of claim 55, wherein one or more of the therapeutic agent(s) includes at least one of a small molecule, a biologic, a hormone, an antibiotic and/or an anti-viral.

60. The composition of claim 55, wherein the hydrogel is capable of releasing the therapeutic agent(s) over a period of time of at least a month.
